SEQUENCE LISTING (308 sequences)

SEQ ID NO: 1 - DNA, 94 bp, Artificial Sequence
  Description of Artificial Sequence Synthetic oligonucleotide
catcgatgctagtcgtaacgatccnnnnnnnnnnnnnnnnnnnnnnnnnnnnnncgagaacgtttctctcctctccctatagtgagtcgtatta

SEQ ID NO: 2 - DNA, 40 bp, Artificial Sequence
  Description of Artificial Sequence Synthetic primer
taatacgactcactatagggagaggagagaaacgttctcg

SEQ ID NO: 3 - DNA, 24 bp, Artificial Sequence
  Description of Artificial Sequence Synthetic primer
catcgatgctagtcgtaacgatcc

SEQ ID NO: 4 - DNA, 62 bp, Artificial Sequence
  Description of Artificial Sequence Synthetic aptamer
gggagaggagagaaacgttctcgattacgcgcgtttgggatcgttacgactagcatcgatga

SEQ ID NO: 5 - DNA, 67 bp, Artificial Sequence
  Description of Artificial Sequence Synthetic aptamer
gggagaggagagaaacgttctcgtacgcgcataccttggtaaggatcgttacgactagcatcgatga

SEQ ID NO: 6 - DNA, 61 bp, Artificial Sequence
  Description of Artificial Sequence Synthetic aptamer
gggagaggagagaaacgttctcgtaagcgcgtattgggatcgttacgactagcatcgatga

SEQ ID NO: 7 - DNA, 63 bp, Artificial Sequence
  Description of Artificial Sequence Synthetic aptamer
gggagaggagagaaacgttctcgtgcgcgcgtctattccgtcatctgatctggggatcgttaa

SEQ ID NO: 8 - DNA, 50 bp, Artificial Sequence
  Description of Artificial Sequence Synthetic aptamer
gggagaggagagaaacgttctcgtgggatcgttacgactagcatcgatga

SEQ ID NO: 9 - DNA, 60 bp, Artificial Sequence
  Description of Artificial Sequence Synthetic aptamer
gggagaggagagaaacgttctcgtacgcgcgctttggatcgttacgactagcatcgatga

SEQ ID NO: 10 - DNA, 55 bp, Artificial Sequence
  Description of Artificial Sequence Synthetic aptamer
gggagaggagagaaacgttctcgtgcgcgcggatcgttacgactagcatcgatga

SEQ ID NO: 11 - DNA, 55 bp, Artificial Sequence; identical to SEQ ID NO: 10
  Description of Artificial Sequence Synthetic aptamer

SEQ ID NO: 12 - DNA, 50 bp, Artificial Sequence
  Description of Artificial Sequence Synthetic aptamer
gggagaggagagaaacgttctcgcgggatcgttacgactagcatcgatga

SEQ ID NO: 13 - DNA, 50 bp, Artificial Sequence; identical to SEQ ID NO: 8
  Description of Artificial Sequence Synthetic aptamer

SEQ ID NO: 14 - DNA, 50 bp, Artificial Sequence; identical to SEQ ID NO: 8
  Description of Artificial Sequence Synthetic aptamer

SEQ ID NO: 15 - DNA, 63 bp, Artificial Sequence
  Description of Artificial Sequence Synthetic aptamer
gggagaggagagaaacgttctcgaatgtggttacgcgcggatcgttacgactagcatcgatga

SEQ ID NO: 16 - DNA, 65 bp, Artificial Sequence
  Description of Artificial Sequence Synthetic aptamer
gggagaggagagaaacgttctcgtatgtcacgaatggcgcggatcgttacgactagcatcgatga

SEQ ID NO: 17 - DNA, 62 bp, Artificial Sequence
  Description of Artificial Sequence Synthetic aptamer
gggagaggagagaaacgttctcgtcggcgcgtcttatggatcgttacgactagcatcgatga

SEQ ID NO: 18 - DNA, 56 bp, Artificial Sequence
  Description of Artificial Sequence Synthetic aptamer
gggagaggagagaaacgttctcgagcgcgtgggatcgttacgactagcatcgatga

SEQ ID NO: 19 - DNA, 71 bp, Artificial Sequence
  Description of Artificial Sequence Synthetic aptamer
gggagaggagagaaacgttctcgtgtacgattgtgcgccgccttgtggatcgttacgactagcatcgatga

SEQ ID NO: 20 - DNA, 78 bp, Artificial Sequence
  Description of Artificial Sequence Synthetic aptamer
gggagaggagagaaacgttctcgtgttctatttcgcgtctcttgttgccttggggatcgttacgactagcatcgatga

SEQ ID NO: 21 - DNA, 66 bp, Artificial Sequence
  Description of Artificial Sequence Synthetic aptamer
gggagaggagagaaacgttctcgttatttagcgcgcgtgtgggatcgttacgactagcatcgatga

SEQ ID NO: 22 - DNA, 74 bp, Artificial Sequence
  Description of Artificial Sequence Synthetic aptamer
gggagaggagagaaacgttctcgtagtcgtccttttagcgcgcgtgtgtggatcgttacgactagcatcgatga

SEQ ID NO: 23 - DNA, 78 bp, Artificial Sequence
  Description of Artificial Sequence Synthetic aptamer
gggagaggagagaaacgttctcgtgtgtgtcaattatcgcgcgtgtgctatggggatcgttacgactagcatcgatga

SEQ ID NO: 24 - DNA, 77 bp, Artificial Sequence
  Description of Artificial Sequence Synthetic aptamer
gggagaggagagaaacgttctcgtgcgcgtcaactgtctgctctgaggtttgggatcgttacgactagcatcgatga

SEQ ID NO: 25 - DNA, 69 bp, Artificial Sequence
  Description of Artificial Sequence Synthetic aptamer
gggagaggagagaaacgttctcgttagactgagcgcgaatctggggatcgttacgactagcatcgatga

SEQ ID NO: 26 - DNA, 78 bp, Artificial Sequence
  Description of Artificial Sequence Synthetic aptamer
gggagaggagagaaacgttctcgttgtccgtctgacgataaacgcggggtcaaggatcgttacgactagcatcgatga

SEQ ID NO: 27 - DNA, 78 bp, Artificial Sequence
  Description of Artificial Sequence Synthetic aptamer
gggagaggagagaaacgttctcgttactacttcgttttgggccttgccgattgggatcgttacgactagcatcgatga

SEQ ID NO: 28 - DNA, 78 bp, Artificial Sequence
  Description of Artificial Sequence Synthetic aptamer
gggagaggagagaaacgttctcgtgcgcgcgactcacttatgccttgttctgcggatcgttacgactagcatcgatga

SEQ ID NO: 29 - DNA, 78 bp, Artificial Sequence
  Description of Artificial Sequence Synthetic aptamer
gggagaggagagaaacgttctcgttcgcgcgctcttaatgttcctccttgcagggatcgttacgactagcatcgatga

SEQ ID NO: 30 - DNA, 78 bp, Artificial Sequence
  Description of Artificial Sequence Synthetic aptamer
gggagaggagagaaacgttctcgtggctgcctgtgtctctcgcgcgcggattgggatcgttacgactagcatcgatga

SEQ ID NO: 31 - DNA, 78 bp, Artificial Sequence
  Description of Artificial Sequence Synthetic aptamer
gggagaggagagaaacgttctcgcgcgacctcatagtccttcctccgtgtatgggatcgttacgactagcatcgatga

SEQ ID NO: 32 - DNA, 78 bp, Artificial Sequence
  Description of Artificial Sequence Synthetic aptamer
gggagaggagagaaacgttctcgtcgacgctctttgtgtgtgaggttgtgtgcggatcgttacgactagcatcgatga

SEQ ID NO: 33 - DNA, 78 bp, Artificial Sequence
  Description of Artificial Sequence Synthetic aptamer
gggagaggagagaaacgttctcgtgtgcgcgctcttactgctattggcttgttggatcgttacgactagcatcgatga

SEQ ID NO: 34 - DNA, 69 bp, Artificial Sequence
  Description of Artificial Sequence Synthetic aptamer
gggagaggagagaaacgttctcgttacattgcgacgcgcggatgggatcgttacgactagcatcgatga

SEQ ID NO: 35 - DNA, 78 bp, Artificial Sequence
  Description of Artificial Sequence Synthetic aptamer
gggagaggagagaaacgttctcgtgagccctatgtcctcctttcgtctgtccgggatcgttacgactagcatcgatga

SEQ ID NO: 36 - DNA, 72 bp, Artificial Sequence
  Description of Artificial Sequence Synthetic aptamer
gggagaggagagaaacgttctcgttcgcgcgctatttccttcgactgggatcgttacgactagcatcgatga

SEQ ID NO: 37 - DNA, 73 bp, Artificial Sequence
  Description of Artificial Sequence Synthetic aptamer
gggagaggagagaaacgttctcgttcgcgcgctttcgatttgcccttgggatcgttacgactagcatcgatga

SEQ ID NO: 38 - DNA, 78 bp, Artificial Sequence
  Description of Artificial Sequence Synthetic aptamer
gggagaggagagaaacgttctcgtgtgtgcgcgcggatggatatgattgcctgggatcgttacgactagcatcgatga

SEQ ID NO: 39 - DNA, 78 bp, Artificial Sequence
  Description of Artificial Sequence Synthetic aptamer
gggagaggagagaaacgttctcgtatgtgttccgtatattccgtccgccttggggatcgttacgactagcatcgatga

SEQ ID NO: 40 - DNA, 78 bp, Artificial Sequence
  Description of Artificial Sequence Synthetic aptamer
gggagaggagagaaacgttctcgagagcgtagtccgtcgagtatcgttcccagggatcgttacgactagcatcgatga

SEQ ID NO: 41 - DNA, 78 bp, Artificial Sequence
  Description of Artificial Sequence Synthetic aptamer
gggagaggagagaaacgttctcgttcgcgcgacttatatttgcctccgacttgggatcgttacgactagcatcgatga

SEQ ID NO: 42 - DNA, 72 bp, Artificial Sequence; identical to SEQ ID NO: 36
  Description of Artificial Sequence Synthetic aptamer

SEQ ID NO: 43 - DNA, 78 bp, Artificial Sequence
  Description of Artificial Sequence Synthetic aptamer
gggagaggagagaaacgttctcgtgtgcgcgtcctgaaattgtcctatgtgtaggatcgttacgactagcatcgatga

SEQ ID NO: 44 - DNA, 78 bp, Artificial Sequence
  Description of Artificial Sequence Synthetic aptamer
gggagaggagagaaacgttctcgttgattgacgcgcgacatacctaacttgtgggatcgttacgactagcatcgatga

SEQ ID NO: 45 - DNA, 78 bp, Artificial Sequence
  Description of Artificial Sequence Synthetic aptamer
gggagaggagagaaacgttctcgatgatacgcacgccttgttatccgacgatgggatcgttacgactagcatcgatga

SEQ ID NO: 46 - DNA, 78 bp, Artificial Sequence
  Description of Artificial Sequence Synthetic aptamer
gggagaggagagaaacgttctcgtagaaggctgacgtccgatatcgaacgcggggatcgttacgactagcatcgatga

SEQ ID NO: 47 - DNA, 74 bp, Artificial Sequence
  Description of Artificial Sequence Synthetic aptamer
gggagaggagagaaacgttctcgtcgcttcacattcgcacgcctcctggggatcgttacgactagcatcgatga

SEQ ID NO: 48 - DNA, 78 bp, Artificial Sequence
  Description of Artificial Sequence Synthetic aptamer
gggagaggagagaaacgttctcgtcnatgtgactaaggatgtacgccgcgtgtggatcgttacgactagcatcgatga

SEQ ID NO: 49 - DNA, 55 bp, Artificial Sequence; identical to SEQ ID NO: 10
  Description of Artificial Sequence Synthetic aptamer

SEQ ID NO: 50 - DNA, 78 bp, Artificial Sequence
  Description of Artificial Sequence Synthetic aptamer
gggagaggagagaaacgttctcgtgcgcgccttctgttcatctgcgttccctgggatcgttacgactagcatcgatga

SEQ ID NO: 51 - DNA, 78 bp, Artificial Sequence
  Description of Artificial Sequence Synthetic aptamer
gggagaggagagaaacgttctcgccggattccttcgtctctaatgtgcgcctgggatcgttacgactagcatcgatga

SEQ ID NO: 52 - DNA, 78 bp, Artificial Sequence
  Description of Artificial Sequence Synthetic aptamer
gggagaggagagaaacgttctcgtccttcggttctcttgtgattgcgcgtgtgggatcgttacgactagcatcgatga

SEQ ID NO: 53 - DNA, 78 bp, Artificial Sequence
  Description of Artificial Sequence Synthetic aptamer
gggagaggagagaaacgttctcgatcgcgcgtgcctactttatgcttccctggggatcgttacgactagcatcgatga

SEQ ID NO: 54 - DNA, 78 bp, Artificial Sequence
  Description of Artificial Sequence Synthetic aptamer
gggagaggagagaaacgttctcgtatgcgcgcgtgacaaactatggctgtgtgggatcgttacgactagcatcgatga

SEQ ID NO: 55 - DNA, 78 bp, Artificial Sequence
  Description of Artificial Sequence Synthetic aptamer
gggagaggagagaaacgttctcgtgtgcgcgcgctctatttttcggtcgtttgggatcgttacgactagcatcgatga

SEQ ID NO: 56 - DNA, 78 bp, Artificial Sequence
  Description of Artificial Sequence Synthetic aptamer
gggagaggagagaaacgttctcgtaagcgcgcgcctctgtgtgggttattgaaggatcgttacgactagcatcgatga

SEQ ID NO: 57 - DNA, 77 bp, Artificial Sequence
  Description of Artificial Sequence Synthetic aptamer
gggagaggagagaaacgttctcggaagcgcgcgcctttatcctgtgttcgacggatcgttacgactagcatcgatga

SEQ ID NO: 58 - DNA, 77 bp, Artificial Sequence; identical to SEQ ID NO: 57
  Description of Artificial Sequence Synthetic aptamer

SEQ ID NO: 59 - DNA, 78 bp, Artificial Sequence
  Description of Artificial Sequence Synthetic aptamer
gggagaggagagaaacgttctcgcgcgctcgtcaattacctcctatcttcgacggatcgttacgactagcatcgatga

SEQ ID NO: 60 - DNA, 78 bp, Artificial Sequence
  Description of Artificial Sequence Synthetic aptamer
gggagaggagagaaacgttctcgcatgagtgagccgcggtaagtgcccctattggatcgttacgactagcatcgatga

SEQ ID NO: 61 - DNA, 77 bp, Artificial Sequence
  Description of Artificial Sequence Synthetic aptamer
gggagaggagagaaacgttctcgtgtgagtcattccgtgtgttagggcccttggatcgttacgactagcatcgatga

SEQ ID NO: 62 - DNA, 66 bp, Artificial Sequence
  Description of Artificial Sequence Synthetic aptamer
gggagaggagagaaacgttctcgttgaatgacgcgcttttgggatcgttacgactagcatcgatga

SEQ ID NO: 63 - DNA, 60 bp, Artificial Sequence
  Description of Artificial Sequence Synthetic aptamer
gggagaggagagaaacgttctcgttggcgcgtttgggatcgttacgactagcatcgatga

SEQ ID NO: 64 - DNA, 78 bp, Artificial Sequence
  Description of Artificial Sequence Synthetic aptamer
gggagaggagagaaacgttctcgttggcgcgtaaaaaatgtttcctgttttgcggatcgttacgactagcatcgatga

SEQ ID NO: 65 - DNA, 78 bp, Artificial Sequence
  Description of Artificial Sequence Synthetic aptamer
gggagaggagagaaacgttctcgtatgcgcgctatttgatacctgttgccgatggatcgttacgactagcatcgatga

SEQ ID NO: 66 - DNA, 78 bp, Artificial Sequence
  Description of Artificial Sequence Synthetic aptamer
gggagaggagagaaacgttctcgtgtatgatgaagcgcgactacttgtgcctaggatcgttacgactagcatcgatga

SEQ ID NO: 67 - DNA, 73 bp, Artificial Sequence
  Description of Artificial Sequence Synthetic aptamer
gggagaggagagaaacgttctcggaagcgcgcgtctttgagtcatgtgggatcgttacgactagcatcgatga

SEQ ID NO: 68 - DNA, 78 bp, Artificial Sequence
  Description of Artificial Sequence Synthetic aptamer
gggagaggagagaaacgttctcgtgtattgccttcctcttatgtgcgcctctgggatcgttacgactagcatcgatga

SEQ ID NO: 69 - DNA, 78 bp, Artificial Sequence
  Description of Artificial Sequence Synthetic aptamer
gggagaggagagaaacgttctcggtggtgatgatctttggcttctcgcgtgtgggatcgttacgactagcatcgatga

SEQ ID NO: 70 - DNA, 76 bp, Artificial Sequence
  Description of Artificial Sequence Synthetic aptamer
gggagaggagagaaacgttctcggttgtaatgactttgattccgcgcgtgaggatcgttacgactagcatcgatga

SEQ ID NO: 71 - DNA, 76 bp, Artificial Sequence
  Description of Artificial Sequence Synthetic aptamer
gggagaggagagaaacgttctcgattgcgcgctttgtgtcccgtgtctataggatcgttacgactagcatcgatga

SEQ ID NO: 72 - DNA, 75 bp, Artificial Sequence
  Description of Artificial Sequence Synthetic aptamer
gggagaggagagaaacgttctcgtggcctaatatgcctctcgagtcacacggatcgttacgactagcatcgatga

SEQ ID NO: 73 - DNA, 77 bp, Artificial Sequence
  Description of Artificial Sequence Synthetic aptamer
gggagaggagagaaacgttctcgtgggcctgtgttcgcgtcttcaagcttcaggatcgttacgactagcatcgatga

SEQ ID NO: 74 - DNA, 78 bp, Artificial Sequence
  Description of Artificial Sequence Synthetic aptamer
gggagaggagagaaacgttctcgngngcccnnnatntnnnnnnnncgagnttgggatcgttacgactagcatcgatga

SEQ ID NO: 75 - DNA, 78 bp, Artificial Sequence
  Description of Artificial Sequence Synthetic aptamer
gggagaggagagaaacgttctcgntggcncncnnncttcttcnngannncnagggatcgttacgactagcatcgatga

SEQ ID NO: 76 - DNA, 78 bp, Artificial Sequence
  Description of Artificial Sequence Synthetic aptamer
gggagaggagagaaacgttctcgtgtgcgccttgttgacttcccttgtttgacggatcgttacgactagcatcgatga

SEQ ID NO: 77 - DNA, 78 bp, Artificial Sequence
  Description of Artificial Sequence Synthetic aptamer
gggagaggagagaaacgttctcgtgttttagccgccttacactccgagaagatggatcgttacgactagcatcgatga

SEQ ID NO: 78 - DNA, 78 bp, Artificial Sequence
  Description of Artificial Sequence Synthetic aptamer
gggagaggagagaaacgttctcgcgcgccgatcttatccttccttcgtgatccggatcgttacgactagcatcgatga

SEQ ID NO: 79 - DNA, 78 bp, Artificial Sequence
  Description of Artificial Sequence Synthetic aptamer
gggagaggagagaaacgttctcgtaacttagaccttctgtttgcgcgcggctcggatcgttacgactagcatcgatga

SEQ ID NO: 80 - DNA, 77 bp, Artificial Sequence
  Description of Artificial Sequence Synthetic aptamer
gggagaggagagaaacgttctcgttggcgcgcgtcttgtcccttattcacgcggatcgttacgactagcatcgatga

SEQ ID NO: 81 - DNA, 77 bp, Artificial Sequence; identical to SEQ ID NO: 80
  Description of Artificial Sequence Synthetic aptamer

SEQ ID NO: 82 - DNA, 78 bp, Artificial Sequence
  Description of Artificial Sequence Synthetic aptamer
gggagaggagagaaacgttctcgttcgcgcgcgattcctattgtccttgttgcggatcgttacgactagcatcgatga

SEQ ID NO: 83 - DNA, 77 bp, Artificial Sequence
  Description of Artificial Sequence Synthetic aptamer
gggagaggagagaaacgttctcgatgtcgcgcctccttgtgtccctgtgtccggatcgttacgactagcatcgatga

SEQ ID NO: 84 - DNA, 77 bp, Artificial Sequence
  Description of Artificial Sequence Synthetic aptamer
gggagaggagagaaacgttctcgtgttcgcgcgccttgtccttgtttatgccggatcgttacgactagcatcgatga

SEQ ID NO: 85 - DNA, 78 bp, Artificial Sequence
  Description of Artificial Sequence Synthetic aptamer
gggagaggagagaaacgttctcgtctttggcctcttattacctctcgaagtacggatcgttacgactagcatcgatga

SEQ ID NO: 86 - DNA, 77 bp, Artificial Sequence
  Description of Artificial Sequence Synthetic aptamer
gggagaggagagaaacgttctcgttcgcgcctaattgcttctccgactgcgtggatcgttacgactagcatcgatga

SEQ ID NO: 87 - DNA, 78 bp, Artificial Sequence
  Description of Artificial Sequence Synthetic aptamer
gggagaggagagaaacgttctcgtgtgcgcgcgccttattgttcccttggcacggatcgttacgactagcatcgatga

SEQ ID NO: 88 - DNA, 78 bp, Artificial Sequence
  Description of Artificial Sequence Synthetic aptamer
gggagaggagagaaacgttctcggtggctatcattctttttctgggcgctttaggatcgttacgactagcatcgatga

SEQ ID NO: 89 - DNA, 78 bp, Artificial Sequence
  Description of Artificial Sequence Synthetic aptamer
gggagaggagagaaacgttctcgagtgttcgcggcttgtgggatttgtggatgggatcgttacgactagcatcgatga

SEQ ID NO: 90 - DNA, 78 bp, Artificial Sequence
  Description of Artificial Sequence Synthetic aptamer
gggagaggagagaaacgttctcgatgtcgatcctcgcgctttccgtctgtttgggatcgttacgactagcatcgatga

SEQ ID NO: 91 - DNA, 78 bp, Artificial Sequence
  Description of Artificial Sequence Synthetic aptamer
gggagaggagagaaacgttctcgtgcgcgacgcctattgccttacgaccctcaggatcgttacgactagcatcgatga

SEQ ID NO: 92 - DNA, 78 bp, Artificial Sequence; identical to SEQ ID NO: 46
  Description of Artificial Sequence Synthetic aptamer

SEQ ID NO: 93 - DNA, 78 bp, Artificial Sequence
  Description of Artificial Sequence Synthetic aptamer
gggagaggagagaaacgttctcgttggccttctactgacgaccccctgacgacggatcgttacgactagcatcgatga

SEQ ID NO: 94 - DNA, 77 bp, Artificial Sequence; identical to SEQ ID NO: 73
  Description of Artificial Sequence Synthetic aptamer

SEQ ID NO: 95 - DNA, 78 bp, Artificial Sequence
  Description of Artificial Sequence Synthetic aptamer
gggagaggagagaaacgttctcgatgacgcctgttatctgtctctatgcgcgcggatcgttacgactagcatcgatga

SEQ ID NO: 96 - DNA, 76 bp, Artificial Sequence
  Description of Artificial Sequence Synthetic aptamer
gggagaggnnngaaacgttctcgatgtccgcgccttttgtctgtccgcctaggatcgttacgactagcatcgatga

SEQ ID NO: 97 - DNA, 78 bp, Artificial Sequence
  Description of Artificial Sequence Synthetic aptamer
gggagaggagagaaacgttctcgttcgcgcgtatgtattgcctccctaggtccggatcgttacgactagcatcgatga

SEQ ID NO: 98 - DNA, 77 bp, Artificial Sequence
  Description of Artificial Sequence Synthetic aptamer
gggagaggagagaaacgttctcgtatgtcgccttccttccgctcgttccgagggatcgttacgactagcatcgatga

SEQ ID NO: 99 - DNA, 79 bp, Artificial Sequence
  Description of Artificial Sequence Synthetic aptamer
gggagaggagagaaacgttctcgatggcgctctttctactacgttgtccgagggggatcgttacgactagcatcgatga

SEQ ID NO: 100 - DNA, 78 bp, Artificial Sequence
  Description of Artificial Sequence Synthetic aptamer
gggagaggagagaaacgttctcgattaagcgcctttatgttcctctgtgtgtgggatcgttacgactagcatcgatga

SEQ ID NO: 101 - DNA, 78 bp, Artificial Sequence
  Description of Artificial Sequence Synthetic aptamer
gggagaggagagaaacgttctcgatggcgcggcctcgctatcttcgtctgtggggatcgttacgactagcatcgatga

SEQ ID NO: 102 - DNA, 78 bp, Artificial Sequence
  Description of Artificial Sequence Synthetic aptamer
gggagaggagagaaacgttctcgttgtctaatgtcgattttttccgcgcgttgggatcgttacgactagcatcgatga

SEQ ID NO: 103 - DNA, 78 bp, Artificial Sequence
  Description of Artificial Sequence Synthetic aptamer
gggagaggagagaaacgttctcgtcgtcgttcgtccgtcttttgccttccgtgggatcgttacgactagcatcgatga

SEQ ID NO: 104 - DNA, 77 bp, Artificial Sequence
  Description of Artificial Sequence Synthetic aptamer
gggagaggagagaaacgttctcgttcgcgcctttgatgtccttcctccgtgtggatcgttacgactagcatcgatga

SEQ ID NO: 105 - DNA, 76 bp, Artificial Sequence
  Description of Artificial Sequence Synthetic aptamer
gggagaggagagaaacgttctcgatgtccgcgccttttgtctgtccgcctaggatcgttacgactagcatcgatga

SEQ ID NO: 106 - DNA, 78 bp, Artificial Sequence
  Description of Artificial Sequence Synthetic aptamer
gggagaggagagaaacgttctcgttgtcgtcactcaggattcattccctacgcggatcgttacgactagcatcgatga

SEQ ID NO: 107 - DNA, 78 bp, Artificial Sequence; identical to SEQ ID NO: 106
  Description of Artificial Sequence Synthetic aptamer

SEQ ID NO: 108 - DNA, 77 bp, Artificial Sequence
  Description of Artificial Sequence Synthetic aptamer
gggagaggagagaaacgttctcgcgcgcgccatacctccttctttccgttcgggatcgttacgactagcatcgatga

SEQ ID NO: 109 - DNA, 71 bp, Artificial Sequence
  Description of Artificial Sequence Synthetic aptamer
gggagaggagagaaacgttctcgctggcgccgtcactctcatgatgggatcgttacgactagcatcgatga

SEQ ID NO: 110 - DNA, 78 bp, Artificial Sequence
  Description of Artificial Sequence Synthetic aptamer
gggagaggagagaaacgttctcgaacacgcgcgtcttcatgcccctagatccgggatcgttacgactagcatcgatga

SEQ ID NO: 111 - DNA, 78 bp, Artificial Sequence
  Description of Artificial Sequence Synthetic aptamer
gggagaggagagaaacgttctcgttgccgcggtccacaagcctcccttcgttcggatcgttacgactagcatcgatga

SEQ ID NO: 112 - DNA, 70 bp, Artificial Sequence
  Description of Artificial Sequence Synthetic aptamer
gggagaggagagaaacgttctcgttcgcgcgctctgtttgccttgggatcgttacgactagcatcgatga

SEQ ID NO: 113 - DNA, 78 bp, Artificial Sequence
  Description of Artificial Sequence Synthetic aptamer
gggagaggagagaaacgttctcgtgcgcgcgatcttacatgccttcccctcgtggatcgttacgactagcatcgatga

SEQ ID NO: 114 - DNA, 78 bp, Artificial Sequence
  Description of Artificial Sequence Synthetic aptamer
gggagaggagagaaacgttctcggatgcgcgcgatgctctacatgttgacctcggatcgttacgactagcatcgatga

SEQ ID NO: 115 - DNA, 78 bp, Artificial Sequence
  Description of Artificial Sequence Synthetic aptamer
gggagaggagagaaacgttctcggctacgcgcgaattacctcttgttgactgcggatcgttacgactagcatcgatga

SEQ ID NO: 116 - DNA, 78 bp, Artificial Sequence
  Description of Artificial Sequence Synthetic aptamer
gggagaggagagaaacgttctcggacgcgcgccctgattcctccttatgattgggatcgttacgactagcatcgatga

SEQ ID NO: 117 - DNA, 78 bp, Artificial Sequence
  Description of Artificial Sequence Synthetic aptamer
gggagaggagagaaacgttctcgggcgcgagacgatccgctatgatggctgtgggatcgttacgactagcatcgatga

SEQ ID NO: 118 - DNA, 78 bp, Artificial Sequence
  Description of Artificial Sequence Synthetic aptamer
gggagaggagagaaacgttctcgctcgcgtcctcctgattgtcgcctccctacggatcgttacgactagcatcgatga

SEQ ID NO: 119 - DNA, 78 bp, Artificial Sequence
  Description of Artificial Sequence Synthetic aptamer
gggagaggagagaaacgttctcgttcgcgcgacttcttgctttcgatctctggggatcgttacgactagcatcgatga

SEQ ID NO: 120 - DNA, 78 bp, Artificial Sequence
  Description of Artificial Sequence Synthetic aptamer
gggagaggagagaaacgttctcgcgcgctttggcccctcttgtcgtcctcctcggatcgttacgactagcatcgatga

SEQ ID NO: 121 - DNA, 76 bp, Artificial Sequence
  Description of Artificial Sequence Synthetic aptamer
gggagaggagagaaacgttctcgttgtcgcgcgtgttgcttcccttggcgtggatcgttacgactagcatcgatga

SEQ ID NO: 122 - DNA, 77 bp, Artificial Sequence; identical to SEQ ID NO: 86
  Description of Artificial Sequence Synthetic aptamer

SEQ ID NO: 123 - DNA, 78 bp, Artificial Sequence
  Description of Artificial Sequence Synthetic aptamer
gggagaggagagaaacgttctcgtattcgagcgcgcgccgacgcttacagctgggatcgttacgactagcatcgatga

SEQ ID NO: 124 - DNA, 74 bp, Artificial Sequence
  Description of Artificial Sequence Synthetic aptamer
gggagaggagagaaacgttctcgtttgtgacgcgcgacctccgatactgggatcgttacgactagcatcgatga

SEQ ID NO: 125 - DNA, 77 bp, Artificial Sequence
  Description of Artificial Sequence Synthetic aptamer
gggagaggagagaaacgttctcgataatggcgcgacttcgatacccgatgagggatcgttacgactagcatcgatga

SEQ ID NO: 126 - DNA, 78 bp, Artificial Sequence
  Description of Artificial Sequence Synthetic aptamer
gggagaggagagaaacgttctcgctaacgtccgcgcgttcttccgttggctatggatcgttacgactagcatcgatga

SEQ ID NO: 127 - DNA, 78 bp, Artificial Sequence
  Description of Artificial Sequence Synthetic aptamer
gggagaggagagaaacgttctcgttgacggacgaattcttcgacgatggccagggatcgttacgactagcatcgatga

SEQ ID NO: 128 - DNA, 78 bp, Artificial Sequence
  Description of Artificial Sequence Synthetic aptamer
gggagaggagagaaacgttctcgctgtaactttgatcctttgagccgcgtttgggatcgttacgactagcatcgatga

SEQ ID NO: 129 - DNA, 78 bp, Artificial Sequence
  Description of Artificial Sequence Synthetic aptamer
gggagaggagagaaacgttctcgtgtaacaatctggaatcgcgcgccgctatgggatcgttacgactagcatcgatga

SEQ ID NO: 130 - DNA, 78 bp, Artificial Sequence
  Description of Artificial Sequence Synthetic aptamer
gggagaggagagaaacgttctcgcttgtacgatcgacctcgtgcgcgccctatggatcgttacgactagcatcgatga

SEQ ID NO: 131 - DNA, 77 bp, Artificial Sequence
  Description of Artificial Sequence Synthetic aptamer
gggagaggagagaaacgttctcgcgatcgatccatgtcttccttgtgcgcgcggatcgttacgactagcatcgatga

SEQ ID NO: 132 - DNA, 78 bp, Artificial Sequence
  Description of Artificial Sequence Synthetic aptamer
gggagaggagagaaacgttctcgtcgacgatgatcccactatctctcgaacgcggatcgttacgactagcatcgatga

SEQ ID NO: 133 - DNA, 78 bp, Artificial Sequence; identical to SEQ ID NO: 132
  Description of Artificial Sequence Synthetic aptamer

SEQ ID NO: 134 - DNA, 78 bp, Artificial Sequence
  Description of Artificial Sequence Synthetic aptamer
gggagaggagagaaacgttctcgttggagctgaagattgacgattgaccttggggatcgttacgactagcatcgatga

SEQ ID NO: 135 - DNA, 78 bp, Artificial Sequence
  Description of Artificial Sequence Synthetic aptamer
gggagaggagagaaacgttctcgcgggttcacgatcgtcgacgcgtttggctaggatcgttacgactagcatcgatga

SEQ ID NO: 136 - DNA, 72 bp, Artificial Sequence
  Description of Artificial Sequence Synthetic aptamer
gggagaggagagaaacgttctcgtacgtcatgtcactctatacgcgcggatcgttacgactagcatcgatga

SEQ ID NO: 137 - DNA, 72 bp, Artificial Sequence
  Description of Artificial Sequence Synthetic aptamer
nnnancnnncnnnnatcancgcgcgaacntccgagaacgtttctctcctctccctatagtgagtcgnattaa

SEQ ID NO: 138 - DNA, 78 bp, Artificial Sequence
  Description of Artificial Sequence Synthetic aptamer
gggagaggaganaaacgttctcgtctacncncgccttgctaattttgcctcgcggatcgttacnactagcatcgatga

SEQ ID NO: 139 - DNA, 78 bp, Artificial Sequence
  Description of Artificial Sequence Synthetic aptamer
gggagaggagagaaacgttctcgntggcgccctnnnnngnnnngngtnncnngggatcgttacnactagcatcgatga

SEQ ID NO: 140 - DNA, 78 bp, Artificial Sequence
  Description of Artificial Sequence Synthetic aptamer
gggagaggananaaacgttctcgctatngtcnctttcngattgcgcgcgcctgggatcgttacgactagcatcgatga

SEQ ID NO: 141 - DNA, 70 bp, Artificial Sequence
  Description of Artificial Sequence Synthetic aptamer
gggagaggagagaaacgttctcgngcgcgcgttttcccntgtcttggatnnntacgactagcatcnatga

SEQ ID NO: 142 - DNA, 50 bp, Artificial Sequence
  Description of Artificial Sequence Synthetic aptamer
nggnnangaganaaacgttctcgtgggatcgttacnactannntcnatga

SEQ ID NO: 143 - DNA, 72 bp, Artificial Sequence
  Description of Artificial Sequence Synthetic aptamer
ggananaaannnnnncgngcncgcgatttacctttggnttgccttgcggatcgntacgactannatcnatga

SEQ ID NO: 144 - DNA, 77 bp, Artificial Sequence
  Description of Artificial Sequence Synthetic aptamer
gggagaggananaaacgttctcgtgcgcctttgactctgtattctcgacctcnganngttacnactagnntcnatga

SEQ ID NO: 145 - DNA, 78 bp, Artificial Sequence
  Description of Artificial Sequence Synthetic aptamer
gggagaggagagaaacgttctcgttngngcgcgctttttgacccctgccctgnggatcgttacgactanngncgatga

SEQ ID NO: 146 - DNA, 77 bp, Artificial Sequence
  Description of Artificial Sequence Synthetic aptamer
ngganaggagagaaacgttctcgttgacgacnnnnantgangtcttgtgcgcggatcgttncnactagcatcnatga

SEQ ID NO: 147 - DNA, 78 bp, Artificial Sequence
  Description of Artificial Sequence Synthetic aptamer
ngnanaggananaaacgntcncnttnncgcgctcttaatgttcctccttgcangnatcgttacnactancatcnatga

SEQ ID NO: 148 - DNA, 77 bp, Artificial Sequence
  Description of Artificial Sequence Synthetic aptamer
gggagaggagagaaacgttctcgttgacgacnnnnanngaggtcttgngcgcgnatcgnnncgactannnncnatga

SEQ ID NO: 149 - DNA, 67 bp, Artificial Sequence
  Description of Artificial Sequence Synthetic aptamer
gggagaggagagaaacgttctcgtggacattacgcgcgattgggatcgttacgactagcatcgatga

SEQ ID NO: 150 - DNA, 78 bp, Artificial Sequence
  Description of Artificial Sequence Synthetic aptamer
gggagaggagagaaacgttctcgattacgcgcccttgtcgctctcgccgtctgggatcgttacgactagcatcgatga

SEQ ID NO: 151 - DNA, 62 bp, Artificial Sequence
  Description of Artificial Sequence Synthetic aptamer
gggagaggagagaaacgttctcgttggcgcgcgtcttggatcgttacgactagcatcgatga

SEQ ID NO: 152 - DNA, 76 bp, Artificial Sequence
  Description of Artificial Sequence Synthetic aptamer
gggagaggagagaaacgttctcgggcgcgtcntatatgnctnatctcctggggatcgttacnactannntcnatga

SEQ ID NO: 153 - DNA, 78 bp, Artificial Sequence
  Description of Artificial Sequence Synthetic aptamer
gggagaggagagaaacgttctcgtgaaggactgggcgcgattgtcgatgttgtggatcgttacgactagcatcgatga

SEQ ID NO: 154 - DNA, 78 bp, Artificial Sequence
  Description of Artificial Sequence Synthetic aptamer
gggagaggagagaaacgttctcgtatgtgcgcgccttccttgttttccgcttgggatcgttacgactagcatcgatga

SEQ ID NO: 155 - DNA, 76 bp, Artificial Sequence
  Description of Artificial Sequence Synthetic aptamer
gggagaggagagaaacgttctcgttgatatggtttgactttgggcgctactgggatcgttacgactagcatcgatg

SEQ ID NO: 156 - DNA, 61 bp, Artificial Sequence
  Description of Artificial Sequence Synthetic aptamer
gggagaggagagaaacgttctcgttgatgacgcgcgtggatcgttacgactagcatcgatg

SEQ ID NO: 157 - DNA, 66 bp, Artificial Sequence
  Description of Artificial Sequence Synthetic aptamer
gggagaggagagaaacgttctcgtgggtgattgcgcgtttgaggatcgttacgactagcatcgatg

SEQ ID NO: 158 - DNA, 77 bp, Artificial Sequence
  Description of Artificial Sequence Synthetic aptamer
gggagaggagagaaacgttctcgtgggttctgatattgctattgccgcgctaaggatcgttacgactagcatcgatg

SEQ ID NO: 159 - DNA, 77 bp, Artificial Sequence
  Description of Artificial Sequence Synthetic aptamer
gggagaggagagaaacgttctcgatgtcttggaatggaattggccgccttatgggatcgttacgactagcatcgatg

SEQ ID NO: 160 - DNA, 77 bp, Artificial Sequence; identical to SEQ ID NO: 159
  Description of Artificial Sequence Synthetic aptamer

SEQ ID NO: 161 - DNA, 54 bp, Artificial Sequence
  Description of Artificial Sequence Synthetic aptamer
gggagaggagagaaacgttctcgttggcgcggatcgttacgactagcatcgatg

SEQ ID NO: 162 - DNA, 76 bp, Artificial Sequence
  Description of Artificial Sequence Synthetic aptamer
gggagaggagagaaacgttctcggctgtcggaattcttgcgcggtgtcttttggatcgttacgactagcatcgatg

SEQ ID NO: 163 - DNA, 78 bp, Artificial Sequence
  Description of Artificial Sequence Synthetic aptamer
gggagaggagagaaacgttctcgtcattggggtttaattcttgtcgcttctttgggatcgttacgactagcatcgatg

SEQ ID NO: 164 - DNA, 60 bp, Artificial Sequence
  Description of Artificial Sequence Synthetic aptamer
gggagaggagagaaacgttctcgattgcgcgcgataggatcgttacgactagcatcgatg

SEQ ID NO: 165 - DNA, 77 bp, Artificial Sequence
  Description of Artificial Sequence Synthetic aptamer
gggagaggagagaaacgttctcgattgcgtcttggagtgcgcgagataccgacggatcgttacgactagcatcgatg

SEQ ID NO: 166 - DNA, 72 bp, Artificial Sequence
  Description of Artificial Sequence Synthetic aptamer
gggagaggagagaaacgttctcgatgatttgggtcctattacgcgacgggatcgttacgactagcatcgatg

SEQ ID NO: 167 - DNA, 78 bp, Artificial Sequence
  Description of Artificial Sequence Synthetic aptamer
gggagaggagagaaacgttctcgggatataagggtcgtgtcttcttgcgcgtgtggatcgttacgactagcatcgatg

SEQ ID NO: 168 - DNA, 54 bp, Artificial Sequence
  Description of Artificial Sequence Synthetic aptamer
gggagaggagagaaacgttctcgaaggcgcggatcgttacgactagcatcgatg

SEQ ID NO: 169 - DNA, 77 bp, Artificial Sequence
  Description of Artificial Sequence Synthetic aptamer
gggagaggagagaaacgttctcgcgtattggttattccgtcttggctcactgaggatcgttacgactagcatcgatg

SEQ ID NO: 170 - DNA, 77 bp, Artificial Sequence
  Description of Artificial Sequence Synthetic aptamer
gggagaggagagaaacgttctcggatacttatgcgccgtgcctcacgctgaacggatcgttacgactagcatcgatg

SEQ ID NO: 171 - DNA, 77 bp, Artificial Sequence
  Description of Artificial Sequence Synthetic aptamer
gggagaggagagaaacgttctcgtgattaatgacgcgtggctacgacttgaggggatcgttacgactagcatcgatg

SEQ ID NO: 172 - DNA, 70 bp, Artificial Sequence
  Description of Artificial Sequence Synthetic aptamer
gggagaggagagaaacgttctcgatgatttatgcgcgtggaagtggggatcgttacgactagcatcgatg

SEQ ID NO: 173 - DNA, 77 bp, Artificial Sequence
  Description of Artificial Sequence Synthetic aptamer
gggagaggagagaaacgttctcgtacttttggcctcggtgttctcgcgcgttgggatcgttacgactagcatcgatg

SEQ ID NO: 174 - DNA, 60 bp, Artificial Sequence
  Description of Artificial Sequence Synthetic aptamer
gggagaggagagaaacgttctcgttaacgcgcgttgggatcgttacgactagcatcgatg

SEQ ID NO: 175 - DNA, 75 bp, Artificial Sequence
  Description of Artificial Sequence Synthetic aptamer
gggagaggagagaaacgttctcgattgagggacttaaggtgtgcgtgtgtgggatcgttacgactagcatcgatg

SEQ ID NO: 176 - DNA, 59 bp, Artificial Sequence
  Description of Artificial Sequence Synthetic aptamer
gggagaggagagaaacgttctcgaatacgcgcgtgggatcgttacgactagcatcgatg

SEQ ID NO: 177 - DNA, 77 bp, Artificial Sequence
  Description of Artificial Sequence Synthetic aptamer
gggagaggagagaaacgttctcggcgactcagaacttacgcttactgctgtggggatcgttacgactagcatcgatg

SEQ ID NO: 178 - DNA, 72 bp, Artificial Sequence
  Description of Artificial Sequence Synthetic aptamer
gggagaggagagaaacgttctcgaatgaggaaatcacgcgcgctttggggatcgttacgactagcatcgatg

SEQ ID NO: 179 - DNA, 46 bp, Artificial Sequence
  Description of Artificial Sequence Synthetic aptamer
gggagaggagagaaacgttctcggatcgttacgactagcatcgatg

SEQ ID NO: 180 - DNA, 46 bp, Artificial Sequence; identical to SEQ ID NO: 179
  Description of Artificial Sequence Synthetic aptamer

SEQ ID NO: 181 - DNA, 73 bp, Artificial Sequence
  Description of Artificial Sequence Synthetic aptamer
gggagaggagagaaacgttctcgttagactgagtgcacgcgcgggtattggatcgttacgactagcatcgatg

SEQ ID NO: 182 - DNA, 47 bp, Artificial Sequence
  Description of Artificial Sequence Synthetic aptamer
gggagaggagagaaacgttctcgggatcgttacgactagcatcgatg

SEQ ID NO: 183 - DNA, 72 bp, Artificial Sequence
  Description of Artificial Sequence Synthetic aptamer
gggagaggagagaaacgttctcgttgatagatgtggcctggcgcgcgcggatcgttacgactagcatcgatg

SEQ ID NO: 184 - DNA, 66 bp, Artificial Sequence
  Description of Artificial Sequence Synthetic aptamer
gggagaggagagaaacgttctcgatatagaagacatgggcgcggatcgttacgactagcatcgatg

SEQ ID NO: 185 - DNA, 77 bp, Artificial Sequence
  Description of Artificial Sequence Synthetic aptamer
gggagaggagagaaacgttctcgttgatgaaatacgcggttcctctagcgcgcggatcgttacgactagcatcgatg

SEQ ID NO: 186 - DNA, 77 bp, Artificial Sequence
  Description of Artificial Sequence Synthetic aptamer
gggagaggagagaaacgttctcgttgctgacgctaatccttcatactacgcgcggatcgttacgactagcatcgatg

SEQ ID NO: 187 - DNA, 58 bp, Artificial Sequence
  Description of Artificial Sequence Synthetic aptamer
gggagaggagagaaacgttctcgttgacgcgcgcggatcgttacgactagcatcgatg

SEQ ID NO: 188 - DNA, 75 bp, Artificial Sequence
  Description of Artificial Sequence Synthetic aptamer
gggagaggagagaaacgttctcgttgacgtatgacttctattcgcgcgcgcggatcgttacgactagcatcgatg

SEQ ID NO: 189 - DNA, 70 bp, Artificial Sequence
  Description of Artificial Sequence Synthetic aptamer
gggagaggagagaaacgttctcgacaatggccgactaatcacgcgcggatcgttacgactagcatcgatg

SEQ ID NO: 190 - DNA, 77 bp, Artificial Sequence
  Description of Artificial Sequence Synthetic aptamer
gggagaggagagaaacgttctcgttgacaagtgactggcttctacatgcgcgcggatcgttacgactagcatcgatg

SEQ ID NO: 191 - DNA, 77 bp, Artificial Sequence
  Description of Artificial Sequence Synthetic aptamer
gggagaggagagaaacgttctcgtatgtcattggcttctgaatatgttcgcgcggatcgttacgactagcatcgatg

SEQ ID NO: 192 - DNA, 66 bp, Artificial Sequence
  Description of Artificial Sequence Synthetic aptamer
gggagaggagagaaacgttctcgtaattggagactgcgcgtgggatcgttacgactagcatcgatg

SEQ ID NO: 193 - DNA, 78 bp, Artificial Sequence
  Description of Artificial Sequence Synthetic aptamer
gggagaggagagaaacgttctcgttattggggatttctgagatacgaattgcgcggatcgttacgactagcatcgatg

SEQ ID NO: 194 - DNA, 70 bp, Artificial Sequence
  Description of Artificial Sequence Synthetic aptamer
gggagaggagagaaacgttctcgcttacgatgtccgtttgccgcgtggatcgttacgactagcatcgatg

SEQ ID NO: 195 - DNA, 77 bp, Artificial Sequence
  Description of Artificial Sequence Synthetic aptamer
gggagaggagagaaacgttctcgtactgtcgagtgtcctttttatgcgcgcgaggatcgttacgactagcatcgatg

SEQ ID NO: 196 - DNA, 76 bp, Artificial Sequence
  Description of Artificial Sequence Synthetic aptamer
gggagaggagagaaacgttctcgtgattgacgacttctttgacgcgcgctgaggatcgttacgactagcatcgatg

SEQ ID NO: 197 - DNA, 78 bp, Artificial Sequence
  Description of Artificial Sequence Synthetic aptamer
gggagaggagagaaacgttctcgattggcggtcttcttcgtcttgcgcgcgtggggatcgttacgactagcatcgatg

SEQ ID NO: 198 - DNA, 77 bp, Artificial Sequence
  Description of Artificial Sequence Synthetic aptamer
gggagaggagagaaacgttctcgtcgtctagttcgcgcccactgcttcctcaaggatcgttacgactagcatcgatg

SEQ ID NO: 199 - DNA, 77 bp, Artificial Sequence
  Description of Artificial Sequence Synthetic aptamer
gggagaggagagaaacgttctcgtcgaatttcgccttcacatacgcgcctcgaggatcgttacgactagcatcgatg

SEQ ID NO: 200 - DNA, 58 bp, Artificial Sequence
  Description of Artificial Sequence Synthetic aptamer
gggagaggagagaaacgttctcgtacgcgcgtgaggatcgttacgactagcatcgatg

SEQ ID NO: 201 - DNA, 77 bp, Artificial Sequence
  Description of Artificial Sequence Synthetic aptamer
gggagaggagagaaacgttctcgtgagtgcggccttgctgagttcctcgtgtgggatcgttacgactagcatcgatg

SEQ ID NO: 202 - DNA, 73 bp, Artificial Sequence
  Description of Artificial Sequence Synthetic aptamer
gggagaggagagaaacgttctcgttgacgcttgttagttccttcgcgtgggatcgttacgactagcatcgatg

SEQ ID NO: 203 - DNA, 77 bp, Artificial Sequence
  Description of Artificial Sequence Synthetic aptamer
gggagaggagagaaacgttctcggtgacgccttcacttctacttatgcgcgtgggatcgttacgactagcatcgatg

SEQ ID NO: 204 - DNA, 77 bp, Artificial Sequence
  Description of Artificial Sequence Synthetic aptamer
gggagaggagagaaacgttctcgttgtcacgccttaacttcttacctacgcgcggatcgttacgactagcatcgatg

SEQ ID NO: 205 - DNA, 76 bp, Artificial Sequence
  Description of Artificial Sequence Synthetic aptamer
gggagaggagagaaacgttctcgttggccttcggacttggacctgcggcgtgggatcgttacgactagcatcgatg

SEQ ID NO: 206 - DNA, 77 bp, Artificial Sequence
  Description of Artificial Sequence Synthetic aptamer
gggagaggagagaaacgttctcgttgcctttacttgttctttcgcgtgttccgggatcgttacgactagcatcgatg

SEQ ID NO: 207 - DNA, 77 bp, Artificial Sequence
  Description of Artificial Sequence Synthetic aptamer
gggagaggagagaaacgttctcgtgtgccttctgctgactccgcgtgttcctgggatcgttacgactagcatcgatg

SEQ ID NO: 208 - DNA, 75 bp, Artificial Sequence
  Description of Artificial Sequence Synthetic aptamer
gggagaggagagaaacgttctcgttggcggccttcggtatagttattgcgcggatcgttacgactagcatcgatg

SEQ ID NO: 209 - DNA, 77 bp, Artificial Sequence
  Description of Artificial Sequence Synthetic aptamer
gggagaggagagaaacgttctcgtgttttggcgccttctgttttcttgttcgcggatcgttacgactagcatcgatg

SEQ ID NO: 210 - DNA, 58 bp, Artificial Sequence
  Description of Artificial Sequence Synthetic aptamer
gggagaggagagaaacgttctcgttgtcgcgcgcggatcgttacgactagcatcgatg

SEQ ID NO: 211 - DNA, 77 bp, Artificial Sequence
  Description of Artificial Sequence Synthetic aptamer
gggagaggagagaaacgttctcgttgtttcggtcttgctgttgtccttcgcgcggatcgttacgactagcatcgatg

SEQ ID NO: 212 - DNA, 54 bp, Artificial Sequence
  Description of Artificial Sequence Synthetic aptamer
gggagaggagagaaacgttctcgtgcgcgcggatcgttacgactagcatcgatg

SEQ ID NO: 213 - DNA, 77 bp, Artificial Sequence
  Description of Artificial Sequence Synthetic aptamer
gggagaggagagaaacgttctcgatgactgtgtttgccctcatggtgacgcgcggatcgttacgactagcatcgatg

SEQ ID NO: 214 - DNA, 67 bp, Artificial Sequence
  Description of Artificial Sequence Synthetic aptamer
gggagaggagagaaacgttctcgtgggttcgcggctgattttgggatcgttacgactagcatcgatg

SEQ ID NO: 215 - DNA, 77 bp, Artificial Sequence
  Description of Artificial Sequence Synthetic aptamer
gggagaggagagaaacgttctcggtcgcttggttcgggcttattcacgttcgcggatcgttacgactagcatcgatg

SEQ ID NO: 216 - DNA, 77 bp, Artificial Sequence
  Description of Artificial Sequence Synthetic aptamer
gggagaggagagaaacgttctcgtgcgctattgcctttgattgcccgtgtcttggatcgttacgactagcatcgatg

SEQ ID NO: 217 - DNA, 77 bp, Artificial Sequence; identical to SEQ ID NO: 216
  Description of Artificial Sequence Synthetic aptamer

SEQ ID NO: 218 - DNA, 77 bp, Artificial Sequence
  Description of Artificial Sequence Synthetic aptamer
gggagaggagagaaacgttctcgatgactgctatctgcctccgaatgtccgtgggatcgttacgactagcatcgatg

SEQ ID NO: 219 - DNA, 71 bp, Artificial Sequence
  Description of Artificial Sequence Synthetic aptamer
gggagaggagagaaacgttctcgtgactatgtgccgctttccgcgtgggatcgttacgactagcatcgatg

SEQ ID NO: 220 - DNA, 63 bp, Artificial Sequence
  Description of Artificial Sequence Synthetic aptamer
gggagaggagagaaacgttctcgttgattgcgcggtttgggatcgttacgactagcatcgatg

SEQ ID NO: 221 - DNA, 76 bp, Artificial Sequence
  Description of Artificial Sequence Synthetic aptamer
gggagaggagagaaacgttctcgtgtgagtcactgattgccgcgcgcccttgggatcgttacgactagcatcgatg

SEQ ID NO: 222 - DNA, 77 bp, Artificial Sequence
  Description of Artificial Sequence Synthetic aptamer
gggagaggagagaaacgttctcgctattgtcgctttctgattgcgcgcgcctgggatcgttacgactagcatcgatg

SEQ ID NO: 223 - DNA, 70 bp, Artificial Sequence
  Description of Artificial Sequence Synthetic aptamer
gggagaggagagaaacgttctcgtatgtgggcgcgcgacgtacctcggatcgttacgactagcatcgatg

SEQ ID NO: 224 - DNA, 77 bp, Artificial Sequence
  Description of Artificial Sequence Synthetic aptamer
gggagaggagagaaacgttctcgtgtgatggtaatggcgcgcttgtggtcctgggatcgttacgactagcatcgatg

SEQ ID NO: 225 - DNA, 77 bp, Artificial Sequence
  Description of Artificial Sequence Synthetic aptamer
gggagaggagagaaacgttctcgttgttgtggttttgggcgcgcgcttggttgggatcgttacgactagcatcgatg

SEQ ID NO: 226 - DNA, 63 bp, Artificial Sequence
  Description of Artificial Sequence Synthetic aptamer
gggagaggagagaaacgttctcgttgattgtgcgcgcttggatcgttacgactagcatcgatg

SEQ ID NO: 227 - DNA, 64 bp, Artificial Sequence
  Description of Artificial Sequence Synthetic aptamer
gggagaggagagaaacgttctcgttgatggcgcggctttaggatcgttacgactagcatcgatg

SEQ ID NO: 228 - DNA, 77 bp, Artificial Sequence
  Description of Artificial Sequence Synthetic aptamer
gggagaggagagaaacgttctcgtgtgtgtgggtcggcgccctttagagtcgcggatcgttacgactagcatcgatg

SEQ ID NO: 229 - DNA, 71 bp, Artificial Sequence
  Description of Artificial Sequence Synthetic aptamer
gggagaggagagaaacgttctcgtgttggcgtgtacttaatgtcgtgggatcgttacgactagcatcgatg

SEQ ID NO: 230 - DNA, 71 bp, Artificial Sequence; identical to SEQ ID NO: 229
  Description of Artificial Sequence Synthetic aptamer

SEQ ID NO: 231 - DNA, 77 bp, Artificial Sequence
  Description of Artificial Sequence Synthetic aptamer
gggagaggagagaaacgttctcgtgcgtattgtgtcgcgttgtactttggtgtggatcgttacgactagcatcgatg

SEQ ID NO: 232 - DNA, 63 bp, Artificial Sequence
  Description of Artificial Sequence Synthetic aptamer
gggagaggagagaaacgttctcgtgatcgcgcgctttgtggatcgttacgactagcatcgatg

SEQ ID NO: 233 - DNA, 77 bp, Artificial Sequence
  Description of Artificial Sequence Synthetic aptamer
gggagaggagagaaacgttctcgtgttcgcgcgcgattttgttcccgttgtgaggatcgttacgactagcatcgatg

SEQ ID NO: 234 - DNA, 58 bp, Artificial Sequence
  Description of Artificial Sequence Synthetic aptamer
gggagaggagagaaacgttctcgttggcgcggatggatcgttacgactagcatcgatg

SEQ ID NO: 235 - DNA, 76 bp, Artificial Sequence
  Description of Artificial Sequence Synthetic aptamer
gggagaggagagaaacgttctcgttgacatacgcgcgctgacgattacccgaggatcgttacgactagcatcgatg

SEQ ID NO: 236 - DNA, 66 bp, Artificial Sequence
  Description of Artificial Sequence Synthetic aptamer
gggagaggagagaaacgttctcgttgtcgcgcgaatattctgggatcgttacgactagcatcgatg

SEQ ID NO: 237 - DNA, 77 bp, Artificial Sequence
  Description of Artificial Sequence Synthetic aptamer
gggagaggagagaaacgttctcgttgtcttatgccgcgtataattgcctgccaggatcgttacgactagcatcgatg

SEQ ID NO: 238 - DNA, 61 bp, Artificial Sequence
  Description of Artificial Sequence Synthetic aptamer
gggagaggagagaaacgttctcgttaatgacgcgcgtggatcgttacgactagcatcgatg

SEQ ID NO: 239 - DNA, 76 bp, Artificial Sequence
  Description of Artificial Sequence Synthetic aptamer
gggagaggagagaaacgttctcgttacttggattcgcgcgcttcgccttggtggatcgttacgactagcatcgatg

SEQ ID NO: 240 - DNA, 77 bp, Artificial Sequence
  Description of Artificial Sequence Synthetic aptamer
gggagaggagagaaacgttctcgtttgtgaatacgcgcgttgtcccttgagtgggatcgttacgactagcatcgatg

SEQ ID NO: 241 - DNA, 64 bp, Artificial Sequence
  Description of Artificial Sequence Synthetic aptamer
gggagaggagagaaacgttctcgtatgacgcgcgcttggtggatcgttacgactagcatcgatg

SEQ ID NO: 242 - DNA, 61 bp, Artificial Sequence
  Description of Artificial Sequence Synthetic aptamer
gggagaggagagaaacgttctcgtatgcgcgcgctttggatcgttacgactagcatcgatg

SEQ ID NO: 243 - DNA, 77 bp, Artificial Sequence
  Description of Artificial Sequence Synthetic aptamer
gggagaggagagaaacgttctcgctagcggtctgcgctttcgccctttggcacggatcgttacgactagcatcgatg

SEQ ID NO: 244 - DNA, 77 bp, Artificial Sequence
  Description of Artificial Sequence Synthetic aptamer
gggagaggagagaaacgttctcgatggcgcgcgcttttaatttggcccttgttggatcgttacgactagcatcgatg

SEQ ID NO: 245 - DNA, 77 bp, Artificial Sequence
  Description of Artificial Sequence Synthetic aptamer
gggagaggagagaaacgttctcggatactcgtaatagctctatttgccgcgcgggatcgttacgactagcatcgatg

SEQ ID NO: 246 - DNA, 73 bp, Artificial Sequence
  Description of Artificial Sequence Synthetic aptamer
gggagaggagagaaacgttctcgttgaatcgcgctttattctgccgcggggatcgttacgactagcatcgatg

SEQ ID NO: 247 - DNA, 77 bp, Artificial Sequence
  Description of Artificial Sequence Synthetic aptamer
gggagaggagagaaacgttctcgatgatacgcgcctcttgattgcctccgtggggatcgttacgactagcatcgatg

SEQ ID NO: 248 - DNA, 64 bp, Artificial Sequence
  Description of Artificial Sequence Synthetic aptamer
gggagaggagagaaacgttctcgatgatacgcgcttttggggatcgttacgactagcatcgatg

SEQ ID NO: 249 - DNA, 77 bp, Artificial Sequence
  Description of Artificial Sequence Synthetic aptamer
gggagaggagagaaacgttctcgatttacgcgcgcgctcttgcgggttagactggatcgttacgactagcatcgatg

SEQ ID NO: 250 - DNA, 57 bp, Artificial Sequence
  Description of Artificial Sequence Synthetic aptamer
gggagaggagagaaacgttctcgattgcgcgcggatggatcgttacgactagcatcg

SEQ ID NO: 251 - DNA, 67 bp, Artificial Sequence
  Description of Artificial Sequence Synthetic aptamer
gggagaggagagaaacgttctcggaattacgcgcgccgacaagggatcgttacgactagcatcgatg

SEQ ID NO: 252 - DNA, 66 bp, Artificial Sequence
  Description of Artificial Sequence Synthetic aptamer
gggagaggagagaaacgttctcgattacgcgcgctgactctgggatcgttacgactagcatcgatg

SEQ ID NO: 253 - DNA, 77 bp, Artificial Sequence
  Description of Artificial Sequence Synthetic aptamer
gggagaggagagaaacgttctcgaatgcgcgcgtcttcatcgtcgagtctgaaggatcgttacgactagcatcgatg

SEQ ID NO: 254 - DNA, 77 bp, Artificial Sequence
  Description of Artificial Sequence Synthetic aptamer
gggagaggagagaaacgttctcgctgacgtccgcgctttgttctccgaatttgggatcgttacgactagcatcgatg

SEQ ID NO: 255 - DNA, 77 bp, Artificial Sequence
  Description of Artificial Sequence Synthetic aptamer
gggagaggagagaaacgttctcgctcgcgacgcctctattgctgctccttctgggatcgttacgactagcatcgatg

SEQ ID NO: 256 - DNA, 77 bp, Artificial Sequence
  Description of Artificial Sequence Synthetic aptamer
gggagaggagagaaacgttctcgtacacacgcgctacttattgcccgacgctcggatcgttacgactagcatcgatg

SEQ ID NO: 257 - DNA, 77 bp, Artificial Sequence
  Description of Artificial Sequence Synthetic aptamer
gggagaggagagaaacgttctcgactacgcgcgcgccgacttctattgcccggggatcgttacgactagcatcgatg

SEQ ID NO: 258 - DNA, 77 bp, Artificial Sequence
  Description of Artificial Sequence Synthetic aptamer
gggagaggagagaaacgttctcgtctacgcgcgccttgctaattttgcctcgcggatcgttacgactagcatcgatg

SEQ ID NO: 259 - DNA, 69 bp, Artificial Sequence
  Description of Artificial Sequence Synthetic aptamer
gggagaggagagaaacgttctcgtttactgtcgacgatttggcgcggatcgttacgactagcatcgatg

SEQ ID NO: 260 - DNA, 76 bp, Artificial Sequence
  Description of Artificial Sequence Synthetic aptamer
gggagaggagagaaacgttctcgtttacacgcgttgatgactccgccgctatggatcgttacgactagcatcgatg

SEQ ID NO: 261 - DNA, 77 bp, Artificial Sequence
  Description of Artificial Sequence Synthetic aptamer
gggagaggagagaaacgttctcgtacgcgcgacttatcattttctggggctttggatcgttacgactagcatcgatg

SEQ ID NO: 262 - DNA, 54 bp, Artificial Sequence; identical to SEQ ID NO: 212
  Description of Artificial Sequence Synthetic aptamer

SEQ ID NO: 263 - DNA, 77 bp, Artificial Sequence
  Description of Artificial Sequence Synthetic aptamer
gggagaggagagaaacgttctcgtgcgcgctgtcttccccttgtactttccgcggatcgttacgactagcatcgatg

SEQ ID NO: 264 - DNA, 77 bp, Artificial Sequence
  Description of Artificial Sequence Synthetic aptamer
gggagaggagagaaacgttctcgcgcgcgcttgcctctaagtacgtcccctgcggatcgttacgactagcatcgatg

SEQ ID NO: 265 - DNA, 54 bp, Artificial Sequence; identical to SEQ ID NO: 212
  Description of Artificial Sequence Synthetic aptamer

SEQ ID NO: 266 - DNA, 77 bp, Artificial Sequence
  Description of Artificial Sequence Synthetic aptamer
gggagaggagagaaacgttctcgtatgcgcctattcactctcatggacctcgtggatcgttacgactagcatcgatg

SEQ ID NO: 267 - DNA, 76 bp, Artificial Sequence
  Description of Artificial Sequence Synthetic aptamer
gggagaggagagaaacgttctcgtgcgcctttgactctgtattcacgacctcggatcgttacgactagcatcgatg

SEQ ID NO: 268 - DNA, 77 bp, Artificial Sequence
  Description of Artificial Sequence Synthetic aptamer
gggagaggagagaaacgttctcggacgcgcgattttggatatgactgactgtgggatcgttacgactagcatcgatg

SEQ ID NO: 269 - DNA, 60 bp, Artificial Sequence
  Description of Artificial Sequence Synthetic aptamer
gggagaggagagaaacgttctcgcgcgcgaaactatggatcgttacgactagcatcgatg

SEQ ID NO: 270 - DNA, 77 bp, Artificial Sequence
  Description of Artificial Sequence Synthetic aptamer
gggagaggagagaaacgttctcgttgacgcgcgctgtacgctattatacgctgggatcgttacgactagcatcgatg

SEQ ID NO: 271 - DNA, 77 bp, Artificial Sequence
  Description of Artificial Sequence Synthetic aptamer
gggagaggagagaaacgttctcgtcgacgcgcgcgaggtattccgagtacgtgggatcgttacgactagcatcgatg

SEQ ID NO: 272 - DNA, 77 bp, Artificial Sequence
  Description of Artificial Sequence Synthetic aptamer
gggagaggagagaaacgttctcgtgtgtgcgccttctactaatgtttatccggggatcgttacgactagcatcgatg

SEQ ID NO: 273 - DNA, 71 bp, Artificial Sequence
  Description of Artificial Sequence Synthetic aptamer
gggagaggagagaaacgttctcgagtgcgcgcgctaactatttgtccggatcgttacgactagcatcgatg

SEQ ID NO: 274 - DNA, 77 bp, Artificial Sequence
  Description of Artificial Sequence Synthetic aptamer
gggagaggagagaaacgttctcggttgcgcgcgctcttatacgattgtccgtgggatcgttacgactagcatcgatg

SEQ ID NO: 275 - DNA, 69 bp, Artificial Sequence
  Description of Artificial Sequence Synthetic aptamer
gggagaggagagaaacgttctcgtgtgcgcgccttatagtccgtgggatcgttacgactagcatcgatg

SEQ ID NO: 276 - DNA, 77 bp, Artificial Sequence
  Description of Artificial Sequence Synthetic aptamer
gggagaggagagaaacgttctcgtgtgcgcgctctatatactttttccgtctgggatcgttacgactagcatcgatg

SEQ ID NO: 277 - DNA, 77 bp, Artificial Sequence
  Description of Artificial Sequence Synthetic aptamer
gggagaggagagaaacgttctcgatgtcggccgccctttatctcttcacgcgtggatcgttacgactagcatcgatg

SEQ ID NO: 278 - DNA, 76 bp, Artificial Sequence
  Description of Artificial Sequence Synthetic aptamer
gggagaggagagaaacgttctcgatgtcgggtgtcttacttcttatctgcgcggatcgttacgactagcatcgatg

SEQ ID NO: 279 - DNA, 54 bp, Artificial Sequence; identical to SEQ ID NO: 212
  Description of Artificial Sequence Synthetic aptamer

SEQ ID NO: 280 - DNA, 60 bp, Artificial Sequence
  Description of Artificial Sequence Synthetic aptamer
gggagaggagagaaacgttctcgtgcgcgctgatatggatcgttacgactagcatcgatg

SEQ ID NO: 281 - DNA, 77 bp, Artificial Sequence
  Description of Artificial Sequence Synthetic aptamer
gggagaggagagaaacgttctcgtgcgcgcgtgattctttgtcgatttcctggggatcgttacgactagcatcgatg

SEQ ID NO: 282 - DNA, 77 bp, Artificial Sequence
  Description of Artificial Sequence Synthetic aptamer
gggagaggagagaaacgttctcggctgttcttcgcgcgtgttcttgtttttggggatcgttacgactagcatcgatg

SEQ ID NO: 283 - DNA, 73 bp, Artificial Sequence
  Description of Artificial Sequence Synthetic aptamer
gggagaggagagaaacgttctcgcgcgcgtatatgtgtttgccgattgtggatcgttacgactagcatcgatg

SEQ ID NO: 284 - DNA, 77 bp, Artificial Sequence
  Description of Artificial Sequence Synthetic aptamer
gggagaggagagaaacgttctcgtacgcgcgtcttttcagagtttctgcttgtggatcgttacgactagcatcgatg

SEQ ID NO: 285 - DNA, 60 bp, Artificial Sequence
  Description of Artificial Sequence Synthetic aptamer
gggagaggagagaaacgttctcgtgcgcgcgttgatggatcgttacgactagcatcgatg

SEQ ID NO: 286 - DNA, 77 bp, Artificial Sequence
  Description of Artificial Sequence Synthetic aptamer
gggagaggagagaaacgttctcgtgcgcgcgtgttattccttatgctcgaatcggatcgttacgactagcatcgatg

SEQ ID NO: 287 - DNA, 77 bp, Artificial Sequence
  Description of Artificial Sequence Synthetic aptamer
gggagaggagagaaacgttctcgtacgcgcgtgtttgatttacttctgattgcggatcgttacgactagcatcgatg

SEQ ID NO: 288 - DNA, 77 bp, Artificial Sequence
  Description of Artificial Sequence Synthetic aptamer
gggagaggagagaaacgttctcgtcgtggccgctgtgcgcgttcttgtcttgaggatcgttacgactagcatcgatg

SEQ ID NO: 289 - DNA, 71 bp, Artificial Sequence
  Description of Artificial Sequence Synthetic aptamer
gggagaggagagaaacgttctcgatgtctgactgtgcgcgtttgtggggatcgttacgactagcatcgatg

SEQ ID NO: 290 - DNA, 75 bp, Artificial Sequence
  Description of Artificial Sequence Synthetic aptamer
gggagaggagagaaacgttctcgttcgcgtttgtgccttggttctgtttggggatcgttacgactagcatcgatg

SEQ ID NO: 291 - DNA, 77 bp, Artificial Sequence
  Description of Artificial Sequence Synthetic aptamer
gggagaggagagaaacgttctcggacgcgtttgaggggttatcagtttgccacggatcgttacgactagcatcgatg

SEQ ID NO: 292 - DNA, 52 bp, Artificial Sequence
  Description of Artificial Sequence Synthetic aptamer
gggagaggagagaaacgttctcgcgcgcggatcgttacgactagcatcgatg

SEQ ID NO: 293 - DNA, 77 bp, Artificial Sequence
  Description of Artificial Sequence Synthetic aptamer
gggagaggagagaaacgttctcgacgacgcgatgaatccttcttgttttccggggatcgttacgactagcatcgatg

SEQ ID NO: 294 - DNA, 77 bp, Artificial Sequence
  Description of Artificial Sequence Synthetic aptamer
gggagaggagagaaacgttctcgtggctgtgcgcgctttttcgatgtttgatgggatcgttacgactagcatcgatg

SEQ ID NO: 295 - DNA, 70 bp, Artificial Sequence
  Description of Artificial Sequence Synthetic aptamer
gggagaggagagaaacgttctcgcttattgtgcgcgcggatgtgatggatcgttacgactagcatcgatg

SEQ ID NO: 296 - DNA, 77 bp, Artificial Sequence
  Description of Artificial Sequence Synthetic aptamer
gggagaggagagaaacgttctcgtgcgcgcggattctattggctgaggatgacggatcgttacgactagcatcgatg

SEQ ID NO: 297 - DNA, 77 bp, Artificial Sequence
  Description of Artificial Sequence Synthetic aptamer
gggagaggagagaaacgttctcgtggattgcgcgcggctatttgctgagatggggatcgttacgactagcatcgatg

SEQ ID NO: 298 - DNA, 60 bp, Artificial Sequence
  Description of Artificial Sequence Synthetic aptamer
gggagaggagagaaacgttctcgtgagcgcggatgaggatcgttacgactagcatcgatg

SEQ ID NO: 299 - DNA, 74 bp, Artificial Sequence
  Description of Artificial Sequence Synthetic aptamer
gggagaggagagaaacgttctcgttgacgcgcggttgatgtttgcttgatggatcgttacgactagcatcgatg

SEQ ID NO: 300 - DNA, 60 bp, Artificial Sequence; identical to SEQ ID NO: 298
  Description of Artificial Sequence Synthetic aptamer

SEQ ID NO: 301 - DNA, 70 bp, Artificial Sequence
  Description of Artificial Sequence Synthetic aptamer
gggagaggagagaaacgttctcggtgacgcgcgacttgtggttgatggatcgttacgactagcatcgatg

SEQ ID NO: 302 - DNA, 78 bp, Artificial Sequence
  Description of Artificial Sequence Synthetic aptamer
gggagaggagagaaacgttctcgttggcgcgcgattgaggtttccgatgtgtggggatcgttacgactagcatcgatg

SEQ ID NO: 303 - DNA, 75 bp, Artificial Sequence
  Description of Artificial Sequence Synthetic aptamer
gggagaggagagaaacgttctcggaggcgcgctgagtttgacgttgtgtttggatcgttacgactagcatcgatg

SEQ ID NO: 304 - DNA, 61 bp, Artificial Sequence
  Description of Artificial Sequence Synthetic aptamer
gggagaggagagaaacgttctcgtgggcgcgcgttatggatcgttacgactagcatcgatg

SEQ ID NO: 305 - DNA, 77 bp, Artificial Sequence
  Description of Artificial Sequence Synthetic aptamer
gggagaggagagaaacgttctcgtgggtgtgcgcgtgattggtgttgtgcttcggatcgttacgactagcatcgatg

SEQ ID NO: 306 - DNA, 69 bp, Artificial Sequence
  Description of Artificial Sequence Synthetic aptamer
gggagaggagagaaacgttctcgtttgcgaggatccgtgggattgggatcgttacgactagcatcgatg

SEQ ID NO: 307 - DNA, 77 bp, Artificial Sequence
  Description of Artificial Sequence Synthetic aptamer
gggagagganagaaacgttctcgtgtgcgcgcgtccgctacttttgactgcgaggatcgttacgactagcatcgatg

SEQ ID NO: 308 - DNA, 74 bp, Artificial Sequence
  Description of Artificial Sequence Synthetic aptamer
gggagaggagagaaacgttctcgattgcgaaaggggatttggattacggnggatcgttacgactagnatcgatg

(19) United States
(12) Patent Application Publication
(10) Pub. No.: US 2010/0144845 A1
(43) Pub. Date: Jun. 10, 2010

(54) OLIGONUCLEOTIDE SYSTEMS FOR TARGETED INTRACELLULAR DELIVERY

(75) Inventors: Omid C. Farokhzad, Chestnut Hill, MA (US); Etgar Levy-Nissenbaum, Boston, MA (US); Robert S. Langer, Newton, MA (US); Frank Alexis, Brighton, MA (US)

Correspondence Address: Pabst Patent Group LLP, 1545 PEACHTREE STREET NE, SUITE 320, ATLANTA, GA, 30309, US

(73) Assignee: Massachusetts Institute of Technology, Cambridge, MA (US)

(21) Appl. No.: 12/376,005

(22) PCT Filed: Aug. 6, 2007
(86) PCT No.: PCT/US2007/017539

§ 371 Date: Feb. 5, 2010

Related U.S. Application Data
(60) Provisional application No. 60/821,408, filed on Aug. 4, 2006.

(51) Int. Cl. A61K 31/7052; C12Q 1/68; C07H 21/00; C12N 5/00; A61P 35/00

(52) U.S. Cl. 514/44 R; 435/6; 536/22.1; 435/325

(57) ABSTRACT

The present invention provides methods for deriving oligonucleotides for specific internal delivery to one or more target cell types (e.g., cancer cells). The method generally includes selecting at least once with a target cell type to provide a plurality of internalizing oligonucleotides for the target cell type, and in some embodiments, counter-selecting at least once with a non-target cell type to provide a plurality of oligonucleotides that do not bind to features present in the non-target cell type. Therapeutic and diagnostic compositions including the oligonucleotides, and methods of treatment are also provided.

### RELATED APPLICATIONS

This application claims priority to U.S. Provisional Application No. 60/821,408 entitled “Method of isolating nucleic acid ligands that are taken up by cells and uses thereof” filed on Aug. 4, 2006, the contents of which are incorporated herein by reference.

### STATEMENT AS TO SPONSORED RESEARCH

Funding for the work described herein was in part supported by a Department of Defense Prostate Cancer Research Program PC 051156 and by the National Institute of Health grants CA119349 and EB003647.

### BACKGROUND OF THE INVENTION

Chemotherapy is the most utilized treatment for cancer but it can have extreme side effects on patients. Chemotherapeutic agents indiscriminately poison rapidly dividing cells, resulting in damage to both cancerous and normal tissues.

Considerable research efforts have been invested trying to control the delivery of cytotoxic drugs in ways that allow cancer cells to be targeted whilst sheltering healthy tissues from exposure.

Clinical application of such therapies depends not only on the efficacy of new delivery systems but also on their safety and on the ease with which the technologies underlying these systems can be adapted for large scale pharmaceutical production, storage, and distribution of the therapeutic formulations. Thus, an ideal vehicle for the delivery of therapeutic agents into cells and tissues should be highly efficient, safe to use, easy to produce in large quantity and have sufficient stability to be practicable as a pharmaceutical.

Accordingly a need exists for new and practical ways of making reagents suitable for introducing therapeutic agents into cells, in vitro and in vivo, and in particular, for use in developing human therapeutics.

### SUMMARY OF THE INVENTION

The present invention is based, in part, on the discovery that oligonucleotides can be selected for preferentially or specifically internalizing into certain cell types, e.g., cancer cells. The oligonucleotides of the invention can be associated with any of a number of therapeutic agents and employed to selectively target a cell type, for example, a cancer cell, in need of being eradicated.

In one embodiment, the oligonucleotides that have been identified as selectively targeting a certain cell type, for example, a cancer cell, are further modified to transfer a therapeutic agent, for example, a small molecule (e.g., chemotherapeutic), a peptide, a protein, or a nucleic acid agent, into cells, cellular or biological spaces, and/or organisms.

One of the advantages of such embodiments is that therapeutic agents previously effective only in relatively high amounts and/or having undesirable side effects can be therapeutically effective in lower and safer amounts when delivered employing the methods and compositions of the invention. Additionally or alternatively, healthy cells, tissues and/or organs can be protected from the harmful effects of, e.g., cytotoxic agents because they are selectively delivered to the target cells. Thus, e.g., harmful effects of chemotherapeutic agents on healthy cells and tissues can be minimized.

Still further, in another embodiment, the oligonucleotides identified by the selection methods describe herein are suitable for efficiently transferring therapeutic agents into cells previously difficult to target.

Accordingly, the invention has several advantages which include, but are not limited to, the following: providing new methods for identifying oligonucleotide carriers or systems for therapeutic delivery; providing oligonucleotide carriers suitable for selectively targeting or treating cancer cells; and providing oligonucleotide carriers for high efficiency transfer of therapeutic agents into specific cells or cell types.

Recent advances in biotechnology—including the development of novel protein based therapeutics (such as monoclonal antibodies and antibody fragments) and therapeutic nucleic acids (such as siRNAs, aptamers, and antisense oligonucleotides)—have all faced considerable pharmacokinetic and bioavailability challenges. In some embodiments, the compositions and methods of the invention can be employed to minimize or eliminate pharmacokinetic and/or bioavailability challenges by delivery of the therapeutic directly to target cells, tissues or organs.

In some embodiments, the effectiveness of therapeutic agents, e.g., chemotherapy-based drugs, can be improved because the compositions and methods can be employed to preferentially deliver agents to specific cells, cellular compartments, organs or tissues.

Accordingly, in one aspect, the present invention provides methods for deriving an oligonucleotide for specific internal delivery to target cells. In some embodiments, such methods include providing a plurality of oligonucleotides, and selecting at least once with target cells to provide at least one internalizing oligonucleotide. In other embodiments, the methods generally include providing a plurality of oligonucleotides, counter-selecting at least once with a non-target cell type, and selecting at least once with target cells to provide at least one internalizing oligonucleotide. In such methods, at least one oligonucleotide is derived that specifically internalizes into target cells.

In some embodiment, the target cells are cancer cells, including any of the cancers disclosed herein. In some embodiments the target cells are cells associated with any of the disorders or conditions disclosed herein, e.g., HIV-infected cells or cells associated with diabetes or heart disease.

In some embodiments, the methods further include mutagenizing the plurality of internalizing oligonucleotides at least once.

In some embodiments, the plurality of oligonucleotides is 2′-O-methyl-modified RNA oligonucleotides.

In some embodiments, a plurality of oligonucleotides is derived that target a plurality of recognition sites. In some embodiments, a plurality of recognition sites are cell surface prostate cancer tumor antigens.

In some embodiments, the non-target cell types can include, but are not limited to, non-cancer cells, normal prostate epithelia cells, normal prostate epithelia cells, RWPE-1 cells, PrEC cells, benign prostate hyperplasia cells, BPH-1 cells, endothelial cells, HUVEC cells, HAEC cells, and combinations thereof.

In some embodiments, the non-target cell can include, but are not limited to, cancer cells, prostate cancer cells, non-small cell lung cancer cells, breast cancer cells, ovarian cancer cells, PC3 cells, LNCaP cells, SKBR3 cells, SKOV3 cells, virus-infected cells, HIV-infected cells, malaria infected cells, hepatitis-infected cells, and combinations thereof.

In some embodiments, the methods of the present invention include a plurality of consecutive incubations with at least one type of non-cancer cell and collecting unbound oligonucleotides. In other embodiments, the methods of the present invention include a plurality of consecutive incubations with at least one type of cancer cells and extracting a plurality of internalizing oligonucleotides from the cancer cells.

In some embodiments, the methods of the present invention further include amplifying after counter-selecting or selecting at least once to provide a plurality of amplified oligonucleotides, and counter-selecting or selecting the plurality of amplified oligonucleotides at least once. In some embodiments, the methods of the present invention include counter-selecting at least five times, and selecting at least three times.

In one aspect, the present invention provides an isolated oligonucleotide that specifically internalizes into at least one target cell type. In another aspect, the present invention provides an isolated plurality of oligonucleotides that specifically internalizes into at least one target cell type.

The target cells can be any cell type associated with a disorder or condition disclosed herein.

In some embodiments, the present invention provides an oligonucleotide or plurality of oligonucleotides that specifically internalizes into target cell types such as cancer cells, prostate cancer cells, non-small cell lung cancer cells, PC3 cells, LNCaP cells, virus-infected cells, HIV-infected cells, malaria infected cells, hepatitis-infected cells, and combinations thereof.

In some embodiments, the present invention provides an oligonucleotide capable of internalizing a therapeutic agent into a target cell type, e.g., a cancer cell type. In further embodiments, the present invention provides an oligonucleotide capable of internalizing a nanoparticle comprising a therapeutic agent into a target cell type, e.g., a cancer cell type. In further embodiments, the present invention provides a plurality of oligonucleotides capable of internalizing a nanoparticle comprising a therapeutic agent into a cancer cell. Such oligonucleotides can include, for example, at least one sequence element such as UGCGCGCG, CGCGCG, GCGCGC, CGCCUU, CGCGCC, GUUCGCG, UGUGUG, UGUGCGC, or the RNA or DNA complement of these sequence elements.

In some embodiments, the present invention provides a plurality of oligonucleotides that target a plurality of recognition sites. The plurality of recognition sites can include at least one cell surface antigen, e.g., a cell surface prostate cancer tumor antigen.

In one aspect, the present invention provides compositions for specific internal delivery of a therapeutic agent to target cells, which include a plurality of oligonucleotides that specifically internalizes into target cells and at least one therapeutic agent associated with at least one of the plurality of oligonucleotides. In some embodiments, at least a portion of the therapeutic agents are docked to a portion of the oligonucleotide. In some embodiments, the composition includes a nanoparticle including a plurality of amphiphilic molecules that establish a hydrophobic core and hydrophilic moieties disposed about the core, and wherein at least a portion of the therapeutic agents are at least partially associated with the hydrophobic core and the oligonucleotide is associated with at least one hydrophilic moiety. The therapeutic agents can include, e.g., a chemotherapeutic agent, a cytotoxic agent, or an antiviral agent.

In another aspect, the present invention provides methods of treating any of the disorder or conditions disclosed herein, e.g., cancer, by administering any of the compositions described herein, e.g., compositions for specific internal delivery of a therapeutic agent to cancer cells, such that an effective amount of the therapeutic agent is delivered to a subject in need of treatment. The cancer can be, for example, prostate cancer.

In one aspect, the present invention provides pharmaceutical formulations which include any of the compositions described herein and a pharmaceutically acceptable carrier.

In one aspect, the present invention provides methods for determining nucleic sequence motifs associated with internalization of oligonucleotides into target cell type. The method generally includes providing a plurality of oligonucleotides; counter-selecting at least once with a non-target cell type to provide a plurality of oligonucleotides that do not bind to features present in the non-target cell type; selecting at least once with a target cell type to provide a plurality of internalizing oligonucleotides for the target cell type; determining at least a portion of the nucleic acid sequence of the plurality of internalizing oligonucleotides for the target cell type; and comparing the nucleic acid sequences, thereby determining common nucleic sequence motifs associated with internalization of oligonucleotides into a first cell type but not a second cell type.

Other features and advantages of the invention will be apparent from the following detailed description and claims.

### BRIEF DESCRIPTION OF THE DRAWINGS

FIG. 1 depicts an exemplary method of deriving oligonucleotides for specific internal delivery to cancer cells. FIG. 1b is a graphical depiction of the progress of the selections made in the method of FIG. 1a.

FIG. 2 is a series of digital images depicting exemplary oligonucleotides derived in accordance with the present invention that are selectively internalized by cancer cells (PC3 and LNCaP). The images depict: (a) labeled oligonucleotides; (b) merged signals from the target cells of the nucleus, cytoskeleton, and the oligonucleotides; and (c) a single-cell close-up of the merged signal image.

FIG. 3 is a schematic representation (a) and a digital image (b) of exemplary nanoparticles of the present invention.

FIGS. 4a-d are a series of digital images demonstrating that exemplary oligonucleotides of the present invention are capable of internalizing nanoparticles of the present invention into cancer cells (PC3 and LNCaP). FIG. 4a depicts: (a) fluorescent nanoparticles entering the cells; (b) merged signals from the target cells of the nucleus, cytoskeleton, and the oligonucleotides; and (c) a single-cell close-up of the merged signal image. FIG. 4b is a three dimensional reconstruction of cell images demonstrating that the nanoparticles are inside the cells. FIGS. 4c-d is a series of images demonstrating the specificity of exemplary nanoparticles for the target cells, and that nanoparticles with unselected oligonucleotides do not internalize.

### DETAILED DESCRIPTION OF THE INVENTION

In order to provide a clear understanding of the specification and claims, the following definitions are conveniently provided below.

### DEFINITIONS

The term “oligonucleotide” refers to RNA molecules as well as DNA molecules. The term RNA refers to a polymer of ribonucleotides. The term “DNA” or “DNA molecule” or deoxyribonucleic acid molecule” refers to a polymer of deoxyribonucleotides. DNA and RNA can be synthesized naturally (e.g., by DNA replication or transcription of DNA, respectively). RNA can be post-transcriptionally modified. DNA and RNA can also be chemically synthesized. DNA and RNA can be single-stranded (i.e., ssRNA and ssDNA, respectively), or multi-stranded (e.g., double stranded, i.e., dsRNA and dsDNA, respectively), i.e., duplexed or annealed. The term “oligonucleotides” includes aptamers, i.e., an oligonucleotide that binds a specific target molecule such as a specific receptor. Non-limiting examples of aptamers include RNA aptamers and DNA aptamers.

Oligonucleotides can be selected or manufactured to be of a certain length, e.g., between about 6 and about 1000 bases, between about 8 and about 500 bases, between about 40 and about 100 bases, between about 50 and about 80 bases, or any range or interval thereof.

Any of the foregoing oligonucleotides are suitable to be complexed with a therapeutic agent. It is understood that any of the oligonucleotides of the invention can also be further modified, for example, chemically modified to include modified bases, methyl groups, altered helical structure, and the like. The term “oligonucleotides” includes modified oligonucleotides.

The term “modified oligonucleotide” or “modified nucleic acid(s)” refers to a non-standard nucleotide or nucleic acid, including non-naturally occurring ribonucleotides or deoxyribonucleotides. Preferred nucleotide analogs or nucleic acids are modified at any position so as to alter certain chemical properties, e.g., increase stability of the nucleotide or nucleic acid yet retain its ability to perform its intended function, e.g., have RNAi activity. Examples include methylation at one or more bases, e.g., O-methylation, preferably 2′ O methylation (2′-O-Me).

Additional examples of nucleotide analogs include azacytidine, inosine, isoguanosine, nebularine, pseudouridine, 2,6-diaminopurine, 2-aminopurine, 2-thiothymidine, 3-deaza-5-azacytidine, 2′-deoxyuridine, 3-nitropyrrole, 4-methylindole, 4-thiouridine, 4-thiothymidine, 2-aminoadenosine, 2-thiothymidine, 2-thiouridine, 5-bromocytidine, 5-iodouridine, nosine, 6-azauridine, 6-chloropurine, 7-deazaadenosine, 7-deazaguanosine, 8-azaadenosine, 8-azidoadenosine, benziinidazole, M1-methyladenosine, pyrrolo-pyrimidine, 2-amino-6-chloropurine, 3-methyl adenosine, 5-propynylcytidine, 5-propynyluridine, 5-bromouridine, 5-fluorouridine, 5-methylcytidine, 7-deazaadenosine, 7-deazaguanosine, 8-oxoadenosine, 8-oxoguanosine, O(6)-methylguanine, 2-thiocytidine, etc.), chemically or biologically modified bases (e.g., methylated bases), intercalated bases, modified sugars (e.g., 2′-fluororibose, 2′-aminoribose, 2′-azidoribose, 2′-O-methylribose, L-enantiomeric nucleosides arabinose, hexose, etc.), modified phosphate moieties (e.g., phosphorothioates or 5′-N-phosphoramidite linkages), and/or other naturally and non-naturally occurring bases substitutable into the polymer including substituted and unsubstituted aromatic moieties.

The phrase “specific internal delivery” refers to delivery of a molecule which is capable of being internalized by at least one cell type or target cell type more efficiently than at least one non-target cell type. The specificity of delivery may be absolute such that a molecule is capable of being internalized by one cell type but not another. Alternatively, the molecule may be internalized several fold (e.g., 0.1, 0.3, 0.5, 1, 2, 3, 4, 5, 6, 7, 8, 9, 10, 15, 20, 30, 40, 50, 100, 1000, 10 000, 100 000 fold) better into one cell type than another. Yet another metric of specific internal delivery is that the molecule is found in an elevated percentage of cells, for example, at about 5%, 10%, 20%, 30%, 40% or about 50%, or more, or an interval or range thereof.

The phrase “derived from” refers to identifying an oligonucleotide with a desired feature using the methods of the invention. Such desired features include, functional features (e.g., the ability to be internalized by a specific cell type) and/or structural features (e.g., sequence motifs or specific nucleic acids associated with a specific functional feature). “Derived from” also encompasses identifying an oligonucleotide with a desired feature by mutagenesis (such that the nucleic acid sequence of the oligonucleotide is altered) or chemical modification of one or more oligonucleotides.

The phrase “oligonucleotides that do not bind to features present in a non-target cell type” refers to oligonucleotides that do not efficiently associate with non-target cell type features, e.g., by internalization or affinity to a surface feature. The oligonucleotides may be completely unable to associate with non-target cell type features such that the affinity for non-target cells is zero. Alternatively, the oligonucleotides may associate with non-target cell type features several fold less efficiently or with a lower affinity than with target cells (e.g., 0.5, 1, 2, 3, 4, 5, 6, 7, 8, 9, 10, 15, 20, 30, 40, 50, 100, 1000, 10 000, 100 000 fold).

The term “recognition sites” refers to sites associated with uptake or internalization, including but not limited to cell surface receptors. Exemplary recognition sites include, but are not limited to, proteins, carbohydrates, membrane lipids, cholesterol or any combination thereof. “Recognition sites” are not limited to binding sites on individual or collections of molecules, the term also refers to broad structural aspects of a cell (e.g. lipid rafts or cavaeolae) or cellular processes (e.g. endocytosis) that allow selective internalization of oligonucleotides into a target cell type but not a non-target cell-type.

The term “therapeutic agent” refers to any agent that, when administered to a cell, tissue, or subject, has a therapeutic and/or diagnostic effect and/or elicits a desired biological and/or pharmacological effect. Therapeutic agents include small molecules (both synthetic and natural), peptides, proteins (including antigen binding molecules), nucleic acids (plasmids, RNA interference agents, antisense agents), chemotherapeutic agents, radioactive agents, lipid-based agents, carbohydrate-based agents, and the like. The therapeutic agents can be mixed, formulated, and/or linked to oligonucleotides using standard methodologies and/or chemistries. The therapeutic agents so linked can also be referred to as conjugates or payloads.

The term “imaging agent” refers to any agent that is useful for imaging purposes (e.g., diagnostic purposes). Example of imaging agents include, e.g., a fluorescent molecule, a radioactive molecule (e.g., comprising a radioisotope), a contrast agent, a lithographic agent, an agent sensitive to ultraviolet light, or an agent sensitive to visible light. Compositions employing an imaging agent can be used, e.g., to identify the location, size or other information regarding tumors. Such information can be used in methods for diagnosis and/or for treatment, e.g., to direct surgeries for removal of targeted cells, tissues or organs.

The term “cell type” refers to a cell or population of cells having a distinct set of morphological, biochemical and/or functional characteristics that define that cell type (e.g., the ability to internalize a specific oligonucleotide). The term “cell type” can refer, e.g., to a broad class of cells (e.g., cancer cells, non-cancer cells and nerve cells), a sub-generic class of cells (e.g., prostate cancer cells, HIV-infected cells and breast cancer cells), or a cell line or group of related cell lines (e.g., PC3 and LNCaP).

The term “cell” refers to any eukaryotic cell, e.g., animal cells (e.g., mammalian cells, e.g., human or murine cells), plant cells, and yeast. The term includes cell lines, e.g., mammalian cell lines such as HeLa cells as well as embryonic cells, e.g., embryonic stem cells and collections of cells in the form of, e.g., a tissue.

The term “cancer cell” includes cells possessing characteristics typical of cancer-causing cells, such as uncontrolled proliferation, immortality, metastatic potential, rapid growth and proliferation rate, and certain characteristic morphological features.

The term “non-cancer cell” includes cells possessing characteristics typical of normal cells, such as controlled proliferation, finite life span, non-metastatic, organized histological features or normal or wild type antigen markers, and the like. The term also includes cells that are cell lines but exhibit one or more normal or non cancer cell phenotypes or genotypes.

The term “cancer” includes pre-malignant as well as malignant cancers. Cancers include, but are not limited to, prostate, gastric cancer, colorectal cancer, skin cancer, e.g., melanomas or basal cell carcinomas, lung cancer, cancers of the head and neck, bronchus cancer, pancreatic cancer, urinary bladder cancer, brain or central nervous system cancer, peripheral nervous system cancer, esophageal cancer, cancer of the oral cavity or pharynx, liver cancer, kidney cancer, testicular cancer, biliary tract cancer, small bowel or appendix cancer, salivary gland cancer, thyroid gland cancer, adrenal gland cancer, osteosarcoma, chondrosarcoma, cancer of hematological tissues, and the like. “Cancer cells” can be in the form of a tumor, exist alone within a subject (e.g., leukemia cells), or be cell lines derived from a cancer.

The term “kit” is any manufacture (e.g. a package or container) comprising at least one reagent (e.g., unselected or selected oligonucleotides, nucleic acid sequence in formation), the manufacture being promoted, distributed, or sold as a unit for performing the methods of the present invention.

### Overview

The present invention is based, at least in part, on the discovery and development of a unique method for deriving oligonucleotides for specific internal delivery to a target cell type. The method generally includes counter-selecting with non-target cells and selecting with a target cell type. Counter-selecting and selecting can be achieved over a number of iterations, and in any order. By way of example, in one embodiment, a plurality of nucleotides is counter-selected a number of times with non-target cell types (e.g., a plurality of healthy cells) to provide a plurality of oligonucleotides that do not bind to features present in the non-target cell type. Subsequently, the plurality of oligonucleotides are selected a number of times (e.g., with several cancer cell lines) to provide a plurality of internalizing oligonucleotides.

A unique pool of oligonucleotides is thus derived that specifically internalize into a target cell type. The oligonucleotides can be employed in a number of therapeutic and diagnostic compositions and methods, for example to increase the accuracy and efficacy of therapeutic agents and/or protect non-target cell types from harm.

Accordingly, in another aspect, the present invention provides methods and compositions for delivering therapeutic agents (e.g., cytotoxic agents) specifically to a cell type or groups of cell types (e.g., cancer cells). The therapeutic agent can be linked, tethered, docked, or otherwise associated with the oligonucleotides in a number of ways. For example, the therapeutic agent can be incorporated into a nanoparticle and the oligonucleotides associated with the nanoparticle surface. Because the oligonucleotides are derived in a manner not constrained to a specific receptor or transport mechanism, the compositions are capable of internalizing in a robust manner by employing a number of pathways into the target cells. Additionally or alternatively, the compositions can also include more than one therapeutic agent (e.g., more than one antiviral medication) to provide a more robust and efficient therapeutic effect.

In yet another aspect, the present invention provides methods and compositions for intracellular delivery of a diagnostic or imaging agent. The method can be employed to diagnose or identify, e.g., a condition or the progression of a condition, e.g., cancer. Additionally, or alternatively, it can be used for therapeutic purposes, e.g., an image can be used before, after or during surgery for removal or protection of target cells, tissues or organs. In some embodiment, the method can be employed in a method for delivery of a therapeutic agent to a target cell, tissue or organ.

### Oligonucleotides

Oligonucleotides useful for the methods of the invention include both RNA and DNA oligonucleotides and art recognized analogues thereof. Preferred nucleotide analogues include sugar- and/or backbone-modified nucleotides (i.e., include modifications to the phosphate-sugar backbone). For example, the phosphodiester linkages of the oligonucleotide can be modified or the 2′ OH-group can be replaced to include methylation at one or more bases, e.g., O-methylation, preferably 2′ O-methylation (2′-O-Me).

In one embodiment, a plurality of oligonucleotides of the invention may be generated from a library of oligonucleotides. Such libraries are available commercially (e.g. from OPERON BIOTECHNOLOGIES) or can be synthesized ab initio using art recognized methods. A plurality of unselected RNA oligonucleotides can be synthesized by transcription from a starter DNA library. The plurality of unselected oligonucleotides can comprise both stochastic sequence elements and fixed sequence elements. The fixed sequence elements can contain oligonucleotide primer binding sites such that the plurality of unselected oligonucleotides can be amplified by polymerase chain reaction (PCR). In one embodiment, a plurality of oligonucleotides is mutagenized to create greater sequence diversity.

Any art recognized methods of nucleic acid sequence mutagenesis are contemplated. In a preferred embodiment, a plurality of RNA oligonucleotides is amplified using reverse transcription and PCR(RT-PCR) under mutagenic conditions (template DNA=25 μg/μL; MgCl₂=7 mM; Tris=10 mM; KCl=50 mM; primers=2 μM; dCTP & dTTP=1 mM; dGTP & dATP=0.2 mM; enzyme=0.05 U/μL; and MnCl₂=0.5 mM; annealing extended to 3 minutes) to create a plurality of DNA oligonucleotides with additional sequence diversity, and the plurality of DNA oligonucleotides are subsequently transcribed back into a plurality of RNA oligonucleotides.

Oligonucleotides can be isolated from cells using any art recognized means. Commercial kits are available for such separation e.g., QIAQUICK from QIAGEN. The isolated oligonucleotides can be amplified using art recognized means such as RT-PCR. The amplified oligonucleotides can be cloned and their nucleic sequence determined using art recognized means. Examples of oligonucleotides selected to internalize specifically into prostate cancer cells can be found in SEQ ID NOS 4-308. In one embodiment the sequence information is used to inform the design of new oligonucleotides for further selection. In another embodiment the sequence information is used to determine nucleic acid consensus sequences or motifs associated with the ability of the oligonucleotides to internalize into specific cells.

Cells can be separated from the plurality of unbound oligonucleotides by any standard art recognized means such as differential centrifugation, filtration and the like. The separation can be enhanced further by washing cells at least once in a suitable physiological buffer.

To isolate oligonucleotides that have been internalized by a cell, it is necessary to separate said oligonucleotides from those that are merely bound to the exterior of the same cell. To achieve this, cells can be treated with agents to enzymatically or chemically remove the oligonucleotides attached to the cell exterior. Suitable compounds include, but are not limited to, nucleases and proteases. In one embodiment, cells are treated with the protease, trypsin, to remove the exterior portions of cell membrane proteins and hence remove any associated RNA oligonucleotides.

In certain embodiments, therapeutic compositions of the invention include a plurality of oligonucleotides that target a plurality of recognition sites, e.g., recognitions sites that are cell surface protein based, cancer antigens, sugars, fatty acids, membrane cell labels.

In some embodiments, the invention provides compositions that include oligonucleotides that target more than one cell type. In certain embodiments, the cell types are related to different indications or symptoms. Such composition can be employed to target cells associated with the same indication, or indications that are related or otherwise tend to occur together.

### Selection Methodology

The invention provides methods for deriving or identifying oligonucleotides capable of being internalized by cells. A variety of cell types are suitable for use in the methods of the invention. For example, a vertebrate cell, e.g., an avian cell or a mammalian cell (e.g., a murine cell, or a human cell). Preferably, the cell is a mammalian cell, e.g., a human cell. All types of cancer cells are contemplated for use in the methods of the invention, including cells isolated directly from tumors, and established cancer cell lines.

Examples of non-target cell types suitable for use in accordance with the present invention include non-cancer cells, normal prostate epithelia cells, RWPE-1 cells, PrEC cells, benign prostate hyperplasia cells, BPH-1 cells, endothelial cells, HUVEC cells, HAEC cells and combinations thereof.

Examples of target cell types suitable for use in accordance with the present invention include cancer cells, prostate cancer cells, non-small cell lung cancer cells, breast cancer cells, ovarian cancer cells, PC3 cells, LNCaP cells, SKBR3 cells, SKOV3 cells, virus-infected cells, HIV-infected cells, malaria infected cells, hepatitis-infected cells, and combinations thereof.

Additional examples of cell lines that can be employed include, but are not limited to, leukemia cells lines such as CCRF-CEM, HL-60(TB), MOLT-4, RPMI-8226, and A549/ATCC. Exemplary Non-Small Cell Lung cell lines include EKVX, HOP-62, HOP-92, NCI-H226, NCI-H322M, and NCI-H522. Colon cancer cell lines include COLO 205, HCC-2998, HCT-116, HCT-15, HT29, and SW-620. Central nervous systems cancer cell lines include SF-295, SF-539, SNB-75, and U251. Exemplary melanoma cell lines include LOX IMVI, MALME-3M, SK-MEL-28, and UACC-257. Exemplary ovarian cancer cell lines include IGR-OV1, OVCAR-4 and SK-OV-3. Exemplary renal cancer cell lines include A498, CAKI-1, TK-10 and UO-31. Exemplary prostate cancer cell lines include PC-3 and DU-145. Exemplary breast cancer cell lines include MCF7, NCI/ADR-RES, HS 578T, MDA-N and T-47D. Small cell lung lines include DMS 114 and SHP-77.

The invention provides methods for deriving or identifying oligonucleotides capable of being specifically internalized by one cell type but not another. Any two cell types or populations of cells are suitable for use in the methods of the invention. For example, in one embodiment, one cell type is a cancer cell, and the other cell type is a normal cell, thereby allowing selection of oligonucleotides capable of being selectively internalized by cancer cells using the methods of the invention. In another embodiment, one cell type is a cell of a specific tissue, and the other cell type is a cell of one or more different tissue, thereby allowing selection of oligonucleotides capable of being selectively internalized by cells of a specific tissue using the methods of the invention. In another embodiment the second cell type is a pathogen infected cell, and the first cell type is a cell of one or more uninfected cells, thereby allowing selection of oligonucleotides capable of being selectively internalized by pathogen infected cells using the methods of the invention

In one embodiment, oligonucleotide selection is achieved by contacting a plurality of oligonucleotides with a specific cell type or types to allow internalization of a subpopulation of oligonucleotides, isolating cells containing the internalized oligonucleotides from the pool of non-internalized oligonucleotides, and extracting the internalized oligonucleotides from the isolated cells, thereby deriving oligonucleotides capable of internalization into the specific cell type.

In another embodiment, oligonucleotide counter-selection is achieved by contacting a plurality of oligonucleotides with a specific cell type or types to allow binding of a subpopulation of oligonucleotides, isolating the remaining pool of unbound oligonucleotides from the cells, thereby deriving a plurality of oligonucleotides depleted of oligonucleotides capable of internalization into the specific cell type.

In another embodiment, oligonucleotide selection is achieved by counter-selection of oligonucleotides with a non-target cell type or types and selection of oligonucleotides with a target cell type or types, thereby selecting oligonucleotides capable of specific internalization into the target cell types but not the non-target cell types. Accordingly, a plurality of oligonucleotides is contacted with non-target cells thereby depleting from the plurality of oligonucleotides which bind, internalize or otherwise associate with the non-target cells. The depleted plurality of oligonucleotides are then contacted with the target cells to allow internalization of a subpopulation of oligonucleotides, cells containing the internalized oligonucleotides are separated form the non-internalized oligonucleotides, and the internalized oligonucleotides are extracted from the isolated cells, thereby selecting oligonucleotides capable of specific internalization by the target cells.

Alternatively the selection step may precede the counter-selection step. The selection and counter-selection step can be performed multiple times in an iterative manner and in any order or combination. The plurality of oligonucleotides can be amplified and mutagenized using art recognized means between each iterative round of selection if desired. In other embodiments, the selection and counter-selection steps can proceed in any order, including alternating series of selection and counter-selection steps.

In some embodiments, the method includes counter-selecting with 1, 2, 3, 4, 5, 6, 7, 8, or 9 or more non-target cell types and selecting with 1, 2, 3, 4, 5, 6, 7, 8, or 9 or more target cell types. For example, the methods can include counter-selecting with 3 non-target cell types and 2 target cell types. Additionally or alternatively, the method can include 2, 3, 4, 5, 6, 7, 8, 9, 10, 11, 12, 13, or 14 or more selection steps and/or counter-selection steps. For example, the method can include 7 counter-selection steps and 5 selection steps in any order.

The above selection technology can be facilitated through the use of any of a number of art recognized high throughput methodologies, for examples, robotics, FACS, and the like.

### Therapeutic Agents

The identified oligonucleotides of the invention are suitable for being admixed, formulated, conjugated, or linked using known chemistries to facilitate the internalization of a therapeutic agent, conjugate, or payload.

Therapeutic agents include drugs, small molecules, peptides, proteins (including antigen binding molecules), nucleic acids (plasmids, RNA interference agents, antisense agents) and the like.

Non-limiting examples of suitable therapeutic agents include antimicrobial agents, analgesics, antiinflammatory agents, counterirritants, coagulation modifying agents, diuretics, sympathomimetics, anorexics, antacids and other gastrointestinal agents; antiparasitics, antidepressants, antihypertensives, anticholinergics, stimulants, antihormones, central and respiratory stimulants, drug antagonists, lipid-regulating agents, uricosurics, cardiac glycosides, electrolytes, ergot and derivatives thereof, expectorants, hypnotics and sedatives, antidiabetic agents, dopaminergic agents, antiemetics, muscle relaxants, parasympathomimetics, anticonvulsants, antihistamines, beta-blockers, purgatives, antiarrhythmics, contrast materials, radiopharmaceuticals, antiallergic agents, tranquilizers, vasodilators, antiviral agents, and antineoplastic or cytostatic agents or other agents with anticancer properties, or a combination thereof. Other suitable medicaments may be selected from contraceptives and vitamins as well as micro- and macronutrients.

Still other examples include anti-infectives such as antibiotics and antiviral agents; analgesics and analgesic combinations; anorexics; antiheimintics; antiarthritics; antiasthmatic agents; anticonvulsants; antidepressants; antidiuretic agents; antidiarrheal; antihistaraines; antiinflammatory agents; antimigraine preparations; antinauseants; antineoplastics; antiparkinsonism drugs; antipruritics; antipsychotics; antipyretics, antispasmodics; anticholinergics; sympathomimetics; xanthine derivatives; cardiovascular preparations including calcium channel blockers and beta-blockers such as pindolol and antiarrhythmics; antihypertensives; diuretics; vasodilators including general coronary, peripheral and cerebral; central nervous system stimulants; cough and cold preparations, including decongestants; hormones such as estradiol and other steroids, including corticosteroids; hypnotics; immunosuppressives; muscle relaxants; parasympatholytics; psychostimulants; sedatives; and tranquilizers; and naturally derived or genetically engineered proteins, polysaccharides, glycoproteins, or lipoproteins.

Exemplary therapeutic agents include chemotherapeutic agents such as doxorubicin (adriamycin), gemcitabine (gemzar), daunorubicin, procarbazine, mitomycin, cytarabine, etoposide, methotrexate, 5-fluorouracil (5-FU), vinblastine, vincristine, bleomycin, paclitaxel (taxol), docetaxel (taxotere), aldesleukin, asparaginase, busulfan, carboplatin, cladribine, camptothecin, CPT-11, 10-hydroxy-7-ethylcamptothecin (SN38), dacarbazine, S-I capecitabine, ftorafur, 5′deoxyfluorouridine, UFT, eniluracil, deoxycytidine, 5-azacytosine, 5-azadeoxycytosine, allopurinol, 2-chloro adenosine, trimetrexate, aminopterin, methylene-10-deazaaminopterin (MDAM), oxaplatin, picoplatin, tetraplatin, satraplatin, platinum-DACH, ormaplatin, CI-973, JM-216, and analogs thereof, epirubicin, etoposide phosphate, 9-aminocamptothecin, 10,11-methylenedioxycamptothecin, karenitecin, 9-nitrocamptothecin, TAS 103, vindesine, L-phenylalanine mustard, ifosphamidemefosphamide, perfosfamide, trophosphamide carmustine, semustine, epothilones A-E, tomudex, 6-mercaptopurine, 6-thioguanine, amsacrine, etoposide phosphate, karenitecin, acyclovir, valacyclovir, ganciclovir, amantadine, rimantadine, lamivudine, zidovudine, bevacizumab, trastuzumab, rituximab, 5-Fluorouracil, and combinations thereof.

Specific non-limiting examples of therapeutic agents include doxorubicin, mitomycin, cisplatin, daunorubicin, bleomycin, actinomycin D, neocarzinostatin, carboplatin, stratoplatin, Ara-C. Other examples include Capoten, Monopril, Pravachol, Avapro, Plavix, Cefzil, DuriceiTUltracef, Azactam, Videx, Zerit, Maxipime, VePesid, Paraplatin, Platinol, Taxol, UFT, Buspar, Serzone, Stadol NS, Estrace, Glucophage (Bristol-Myers Squibb); Ceclor, Lorabid, Dynabac, Prozac, Darvon, Permax, Zyprexa, Humalog, Axid, Gemzar, Evista (Eli Lily); Vasotec/Vaseretic, Mevacor, Zocor, Prinivil/Prinizide, Plendil, Cozaar/Hyzaar, Pepcid, Prilosec, Primaxin, Noroxin, Recombivax HB, Varivax, Timoptic/XE, Trusopt, Proscar, Fosamax, Sinemet, Crixivan, Propecia, Vioxx, Singulair, Maxalt, Ivermectin (Merck & Co.); Diflucan, Unasyn, Sulperazon, Zithromax, Trovan, Procardia XL, Cardura, Norvasc, Dofetilide, Feldene, Zoloft, Zeldox, Glucotrol XL, Zyrtec, Eletriptan, Viagra, Droloxifene, Aricept, Lipitor (Pfizer); Vantin, Rescriptor, Vistide, Genotropin, Micronase/GlynVGlyb., Fragmin, Total Medrol, Xanax/alprazolam, Sermion, Halcion/triazolam, Freedox, Dostinex, Edronax, Mirapex, Pharmorubicin, Adriamycin, Camptosar, Remisar, Depo-Provera, Caverject, Detrusitol, Estring, Healon, Xalatan, Rogaine (Pharmacia & Upjohn); Lopid, Accrupil, Dilantin, Cognex, Neurontin, Loestrin, Dilzem, Fempatch, Estrostep, Rezulin, Lipitor, Omnicef, FemHRT, Suramin, or Clinafloxacin (Warner Lambert).

As another example, if the target cell is a cancer cell, then the therapeutic agent may be an anti-cancer drug such as 20-epi-1, 25 dihydroxyvitamin D3,4-ipomeanol, 5-ethynyluracil, 9-dihydrotaxol, abiraterone, acivicin, aclarubicin, acodazole hydrochloride, acronine, acylfulvene, adecypenol, adozelesin, aldesleukin, all-tk antagonists, altretamine, ambamustine, ambomycin, ametantrone acetate, amidox, amifostine, aminoglutethimide, aminolevulinic acid, amrubicin, amsacrine, anagrelide, anastrozole, andrographolide, angiogenesis inhibitors, antagonist D, antagonist G, antarelix, anthramycin, anti-dorsalizing morphogenetic protein-1, antiestrogen, antineoplaston, antisense oligonucleotides, aphidicolin glycinate, apoptosis gene modulators, apoptosis regulators, apurinic acid, ARA-CDP-DL-PTBA, arginine deaminase, asparaginase, asperlin, asulacrine, atamestane, atrimustine, axinastatin 1, axinastatin 2, axinastatin 3, azacitidine, azasetron, azatoxin, azatyrosine, azetepa, azotomycin, baccatin III derivatives, balanol, batimastat, benzochlorins, benzodepa, benzoylstaurosporine, beta lactam derivatives, beta-alethine, betaclamycin B, betulinic acid, BFGF inhibitor, bicalutamide, bisantrene, bisantrene hydrochloride, bisaziridinylspermine, bisnafide, bisnafide dimesylate, bistratene A, bizelesin, bleomycin, bleomycin sulfate, BRC/ABL antagonists, breflate, brequinar sodium, bropirimine, budotitane, busulfan, buthionine sulfoximine, cactinomycin, calcipotriol, calphostin C, calusterone, camptothecin derivatives, canarypox IL-2, capecitabine, caracemide, carbetimer, carboplatin, carboxamide-amino-triazole, carboxyamidotriazole, carest M3, carmustine, earn 700, cartilage derived inhibitor, carubicin hydrochloride, carzelesin, casein kinase inhibitors, castanospermine, cecropin B, cedefingol, cetrorelix, chlorambucil, chlorins, chloroquinoxah'ne sulfonamide, cicaprost, cirolemycin, cisplatin, cis-porphyrin, cladribine, clomifene analogs, clotrimazole, collismycin A, collismycin B, combretastatin A4, combretastatin analog, conagenin, crambescidin 816, crisnatol, crisnatol mesylate, cryptophycin 8, cryptophycin A derivatives, curacin A, cyclopentanthraquinones, cyclophosphamide, cycloplatam, cypemycin, cytarabine, cytarabine ocfosfate, cytolytic factor, cytostatic, dacarbazine, dacliximab, dactinomycin, daunorubichi hydrochloride, decitabine, dehydrodidemnin B, deslorelm, dexifosfamide, dexormaplatb, dexrazoxane, dexverapamil, dezaguanine, dezaguanine mesylate, diaziquone, didemnin B, didox, diethyborspermine, dihydro-5-azacytidine, dioxamycin, diphenyl spiromustbe, docetaxel, docosanol, dolasetron, doxifluridine, doxorubicin, doxorubicin hydrochloride, droloxifene, droloxifene citrate, dromostanolone propionate, dronabinol, duazomycin, duocannycin SA, ebselen, ecomustine, edatrexate, edelfosine, edrecolomab, eflornithine, eflornithine hydrochloride, elemene, elsamitrucin, emiterur, enloplatin, enpromate, epipropidine, epirubicin, epirabicin hydrochloride, epristeride, erbulozole, erythrocyte gene therapy vector system, esorubicin hydrochloride, estramustine, estramustine analog, estramustine phosphate sodium, estrogen agonists, 5 estrogen antagonists, etanidazole, etoposide, etoposide phosphate, etoprine, exemestane, fadrozole, fadrozole hydrochloride, fazarabine, fenretinide, filgrastim, finasteride, flavopiridol, flezelastine, floxuridine, fluasterone, fludarabine, fludarabirie phosphate, fluorodaunorunicin hydrochloride, fluorouracil, fluorocitabine, forfenimex, formestane, fosquidone, fostriecin, fostriecin sodium, fotemustine, gadolinium texaphyrin, gallium nitrate, galocitabine, ganirelix, gelatinase inhibitors, gemcitabine, gemcitabine hydrochloride, glutathione inhibitors, hepsulfam, heregulin, hexamethylene bisacetamide, hydroxyurea, hypericin, ibandronic acid, idarubicin, idambicin hydrochloride, idoxifene, idramantone, ifosfamide, ihnofosine, ilomastat, imidazoacridones, imiquimod, immunostimulant peptides, insulin-like growth factor-1receptor inhibitor, interferon agonists, interferon alpha-2A, interferon alpha-2B, interferon alpha-N1, interferon alpha-N3, interferon beta-IA, interferon gamma-IB, interferons, interleukins, iobenguane, iododoxorubicin, iproplatin, irinotecan, irinotecan hydrochloride, iroplact, irsogladine, isobengazole, isohomohalicondrin B, itasetron, jasplakinolide, kahalalide F, lamellarin-N triacetate, lanreotide, lanreotide acetate, leinamycin, lenograstim, lentinan sulfate, leptolstatin, letrozole, leukemia inhibiting factor, leukocyte alpha interferon, leuprolide acetate, leuprolide/estrogen/progesterone, leuprorelin, levamisole, liarozole, liarozole hydrochloride, linear polyamine analog, lipophilic disaccharide peptide, lipophilic platinum compounds, lissoclinamide 7, lobaplatin, lombricine, lometrexol, lometrexol sodium, lomustine, lonidamine, losoxantrone, losoxantrone hydrochloride, lovastatin, loxoribine, lurtotecan, lutetium texaphyrin, lysofylline, lytic peptides, maitansine, mannostatin A, marimastat, masoprocol, maspin, matrilysin inhibitors, matrix metalloproteinase inhibitors, maytansine, mechlorethamine hydrochloride, megestrol acetate, melengestrol acetate, melphalan, menogaril, merbarone, mercaptopurine, meterelin, methioninase, methotrexate, methotrexate sodium, metoclopramide, metoprine, meturedepa, microalgal protein kinase C inhibitors, MIF inhibitor, mifepristone, miltefosine, mirimostim, mismatched double stranded RNA, mitindomide, mitocarcin, mitocromin, mitogillin, mitoguazone, mitolactol, mitomycin, mitomycin, mitomycin analogs, mitonafide, mitosper, mitotane, mitotoxin fibroblast growth factor-saporin, mitoxantrone, mitoxantrone hydrochloride, mofarotene, molgramostim, monoclonal antibody, human chorionic gonadotrophin, monophosphoryl lipid a/mycobacterium cell wall SK, mopidamol, multiple drug resistance gene inhibitor, multiple tumor suppressor 1-based therapy, mustard anticancer agent, mycaperoxide B, mycobacterial cell wall extract, mycophenolic acid, myriaporone, n-acetyldinaline, nafarelin, nagrestip, naloxone/pentazocine, napavin, naphterpin, nartograstim, nedaplatin, nemorubicin, neridronic acid, neutral endopeptidase, nilutamide, nisamycin, nitric oxide modulators, nitroxide antioxidant, nitrullyn, nocodazole, nogalamycin, n-substituted benzamides, O6-benzylguanine, octreotide, okicenone, oligonucleotides, onapristone, ondansetron, oracin, oral cytokine inducer, ormaplatin, osaterone, oxaliplatin, oxaunomycin, oxisuran, paclitaxel, paclitaxel analogs, paclitaxel derivatives, palauamine, palmitoylrhizoxin, pamidronic acid, panaxytriol, panomifene, parabactin, pazelliptine, pegaspargase, peldesine, peliomycin, pentamustine, pentosan polysulfate sodium, pentostatin, pentrozole, peplomycin sulfate, perflubron, perfosfamide, perillyl alcohol, phenazdnomycin, phenylacetate, phosphatase inhibitors, picibanil, pilocarpine hydrochloride, pipobroman, piposulfan, pirarubicin, piritrexim, piroxantrone hydrochloride, placetin A, placetin B, plasminogen activator inhibitor, platinum complex, platinum compounds, platinum-triamine complex, plicamycin, plomestane, porfimer sodium, porfiromycin, prednimustine, procarbazine hydrochloride, propyl bis-acridone, prostaglandin J2, prostatic carcinoma antiandrogen, proteasome inhibitors, protein A-based immune modulator, protein kinase C inhibitor, protein tyrosine phosphatase inhibitors, purine nucleoside phosphorylase inhibitors, puromycin, puromycin hydrochloride, purpurins, pyrazofurin, pyrazoloacridine, pyridoxylated hemoglobin polyoxyethylene conjugate, RAF antagonists, raltitrexed, ramosetron, RAS farnesyl protein transferase inhibitors, RAS inhibitors, RAS-GAP inhibitor, retelliptine demethylated, rhenium RE 186 etidronate, rhizoxin, riboprine, ribozymes, RH retinamide, RNAi, rogletimide, rohitukine, romurtide, roquinimex, rubiginone B1, ruboxyl, safingol, safingol hydrochloride, saintopin, sarcnu, sarcophytol A, sargramostim, SDI1 mimetics, semustine, senescence derived inhibitor 1, sense oligonucleotides, signal transduction inhibitors, signal transduction modulators, simtrazene, single chain antigen binding protein, sizofuran, sobuzoxane, sodium borocaptate, sodium phenylacetate, solverol, somatomedin binding protein, sonermin, sparfosate sodium, sparfosic acid, sparsomycin, spicamycin D, spirogermanium hydrochloride, spiromustine, spiroplatin, splenopentin, spongistatin 1, squalamine, stem cell inhibitor, stem-cell division inhibitors, stipiamide, streptonigrin, streptozocin, stromelysin hihibitors, sulfinosine, sulofenur, superactive vasoactive intestinal peptide antagonist, suradista, suramin, swainsonine, synthetic glycosaminoglycans, talisomycin, tallimustine, tamoxifen methiodide, tauromustine, tazarotene, tecogalan sodium, tegafur, tellurapyrylium, telomerase inhibitors, teloxantrone hydrochloride, temoporfin, temozolomide, teniposide, teroxirone, testolactone, tetrachlorodecaoxide, tetrazomine, thaliblastine, thalidomide, thiamiprine, thiocoraline, thioguanine, thiotepa, thrombopoietin, thrombopoietin mimetic, thymalfasin, thymopoietin receptor agonist, thymotrinan, thyroid stimulating hormone, tiazofurin, tin ethyl etiopurpurin, tirapazamine, titanocene dichloride, topotecan hydrochloride, topsentin, toremifene, toremifene citrate, totipotent stem cell factor, translation inhibitors, trestolone acetate, tretinoin, triacetyluridine, triciribine, triciribine phosphate, trimetrexate, trimetrexate glucuronate, triptorelin, tropisetron, tubulozole hydrochloride, turosteride, tyrosine kinase inhibitors, tyrphostins, UBC inhibitors, ubenimex, uracil mustard, uredepa, urogenital sinus-derived growth inhibitory factor, urokinase receptor antagonists, vapreotide, variolin B, velaresol, veramine, verdins, verteporfin, vinblastine sulfate, vincristine sulfate, vindesine, vindesine sulfate, vinepidine sulfate, vinglycinate sulfate, vinleurosine sulfate, vinorelbine, vinorelbine tartrate, vinrosidine sulfate, vinxaltine, vinzolidine sulfate, vitaxin, vorozole, zanoterone, zeniplatin, zilascorb, zinostatin, zinostatin stimalamer, or zorubicin hydrochloride. Further specific non-limiting examples of drugs that can be included within a particle of the present invention include acebutolol, acetaminophen, acetohydroxamic acid, acetophenazine, acyclovir, adrenocorticoids, allopurinol, alprazolam, aluminum hydroxide, amantadine, ambenonium, amiloride, aminobenzoate potassium, amobarbital, amoxicillin, amphetamine, ampicillin, androgens, anesthetics, anticoagulants, anticonvulsants-dione type, antithyroid medicine, appetite suppressants, aspirin, atenolol, atropine, azatadine, bacampicillin, baclofen, beclomethasone, belladonna, bendroflumethiazide, benzoyl peroxide, benzthiazide, benztropine, betamethasone, betha nechol, biperiden, bisacodyl, bromocriptine, bromodiphenhydramine, brompheniramine, buclizine, bumetanide, busulfan, butabarbital, butaperazine, caffeine, calcium carbonate, captopril, carbamazepine, carbenicillin, carbidopa, levodopa, carbinoxamine inhibitors, carbonic anhydrase, carisoprodol, carphenazine, cascara, cefaclor, cefadroxil, cephalexin, cephradine, chlophedianol, chloral hydrate, chlorambucil, chloramphenicol, chlordiazepoxide, chloroquine, chlorothiazide, chlorotrianisene, chlorpheniramine, chlorpromazine, chlorpropamide, chlorprothixene, chlorthalidone, chlorzoxazone, cholestyramine, cimetidine, cinoxacin, clemastine, clidinium, clindamycin, clofibrate, clomiphere, clonidine, clorazepate, cloxacillin, colochicine, coloestipol, conjugated estrogen, contraceptives, cortisone, cromolyn, cyclacillin, cyclandelate, cyclizine, cyclobenzaprine, cyclophosphamide, cyclothiazide, cycrimine, cyproheptadine, danazol, danthron, dantrolene, dapsone, dextroamphetamine, dexamethasone, dexchlorpheniramine, dextromethorphan, diazepan, dicloxacillin, dicyclomine, diethylstilbestrol, diflunisal, digitalis, diltiazen, dimenhydrinale, dimethindene, diphenhydramine, diphenidol, diphenoxylate & atrophive, diphenylopyraline, dipyradamole, disopyramide, disulfiram, divalporex, docusate calcium, docusate potassium, docusate sodium, doxyloamine, dronabinol ephedrine, epinephrine, ergoloidmesylates, ergonovine, ergotatnine, erythromycins, esterified estrogens, estradiol, estrogen, estrone, estropipute, etharynic acid, ethchlorvynol, ethinyl estradiol, ethopropazine, ethosaximide, ethotoin, fenoprofen, ferrous fumarate, ferrous gluconate, ferrous sulfate, flavoxate, flecamide, fluphenazine, fluprednisolone, flurazepam, folic acid, furosemide, gemfibrozil, glipizide, glyburide, glycopyrrolate, gold compounds, griseofiwin, guaifenesin, guanabenz, guanadrel, guanethidine, halazepam, haloperidol, hetacillin, hexobarbital, hydralazine, hydrochlorothiazide, hydrocortisone (cortisol), hydroflunethiazide, hydroxychloroquine, hydroxyzine, hyoscyamine, ibuprofen, indapamide, indomethacin, insulin, iofoquinol, iron-polysaccharide, isoetharine, isoniazid, isopropamide isoproterenol, isotretinoin, isoxsuprine, kaolin & pectin, ketoconazole, lactulose, levodopa, lincomycin liothyronine, liotrix, lithium, loperamide, lorazepam, magnesium hydroxide, magnesium sulfate, magnesium trisilicate, maprotiline, meclizine, meciofenamate, medroxyproyesterone, melenamic acid, melphalan, mephenyloin, mephobarbital, meprobamate, mercaptopurine, mesoridazine, metaproterenol, metaxalone, methamphetamine, methaqualone, metharbital, methenamine, methicillin, methocarbamol, methotrexate, methsuximide, methyclothinzide, methylcellulose, methyldopa, methylergonovine, methylphenidate, methylprednisolone, methysergide, metoclopramide, metolazone, metoprolol, metronidazole, minoxidil, mitotane, monamine oxidase inhibitors, nadolol, nafcillin, nalidixic acid, naproxen, narcotic analgesics, neomycin, neostigmine, niacin, nicotine, nifedipine, nitrates, nitrofurantoin, nomifensine, norethindrone, norethindrone acetate, norgestrel, nylidrin, nystafin, orphenadrine, oxacillin, oxazepam, oxprenolol, oxymetazoline, oxyphenbutazone, pancrelipase, pantothenic acid, papaverine, para-aminosalicylic acid, paramethasone, paregoric, pemoline, penicillamine, penicillin, penicillin-v, pentobarbital, perphenazine, phenacetin, phenazopyridine, pheniramine, phenobarbital, phenolphthalein, phenprocoumon, phensuximide, phenylbutazone, phenylephrine, phenylpropanolamine, phenyl toloxamme, phenyloin, pilocarpine, pindolol, piper acetazine, piroxicam, poloxamer, polycarbophil calcium, polythiazide, potassium supplements, prazepam, prazosin, prednisolone, prednisone, primidone, probenecid, probucol, procainamide, procarbazine, prochlorperazine, procyclidine, promazine, promethazine, propantheline, propranolol, pseudoephedrine, psoralens, psyllium, pyridostigmine, pyridoxine, pyrilamine, pyrvinium, quinestrol, quinethazone, quinidine, quinine, ranitidine, rauwolfia alkaloids, riboflavin, rifampin, ritodrine, salicylates, scopolamine, secobarbital, senna, sannosides α and β, simethicone, sodium bicarbonate, sodium phosphate, sodium fluoride, spironolactone, sucrulfate, sulfacytine, sulfamethoxazole, sulfasalazine, sulfinpyrazone, sulfisoxazole, sulindac, talbutal, tamazepam, terbutaline, terfenadine, terphinhydrate, tertacyclines, thiabendazole, thiamine, thioridazine, thiothixene, thyroblobulin, thyroid, thyroxine, ticarcillin, timolol, tocainide, tolazamide, tolbutamide, tolmetin trozodone, tretinoin, triamcinolone, trianterene, triazolam, trichlormethiazide, tricyclic antidepressants, tridhexethyl, trifluoperazine, triflupromazine, trihexyphenidyl, trimeprazine, trimethobenzamine, trimethoprim, tripelennamine, triprolidine, valproic acid, verapamil, vitamin A, vitamin B12, vitamin C, vitamin D, vitamin E, vitamin K, xanthine, and the like.

In some embodiments, because therapeutic agents are delivered specifically to target cells, toxicity or other negative attributes of therapeutic agents or combinations of agents are reduced to an acceptable extent or eliminated. In some embodiments, therapeutic agents or combinations of agents that otherwise present unacceptable or undesired side effects or toxicities can now be employed because toxicity is diminished to an acceptable extent or eliminated. Accordingly, in some embodiments, the invention provides a pharmaceutical composition that provides improved safety, reduced toxicity, improved efficacy and/or acceptable side effects, and methods for treating a subject employing such compositions.

In some embodiments, therapeutic agents employed in the compositions of the present invention include mitoxantrone, morpholino-doxorubicin, rhizoxin, cyanomorpholino-doxorubicin, dolastatin-10, echinomycin, combretastatin, calicheamicin, maytansine, DM-1, auristatin E, and related compounds and derivatives.

Therapeutic agents can be associated with the oligonucleotides of the invention employing any number of methods or technologies, e.g., nanoparticles, liposomes, linking moieties, direct covalent bonds, nanoshells, and incorporation of at least part of the therapeutic agent into at least part of the oligonucleotide.

In some embodiments, biodegradable nanoparticles are employed, which can also be employed for controlled release of some or all of the therapeutic agents. As used herein, the term “nanoparticles” refers to particles having an average or mean diameter of less than about 1 micron. In some embodiments, the average or mean diameter of the nanoparticles is be less than about 300 nm, less than about 200 nm, less than about 150 nm, less than about 100 nm, less than about 50 nm, less than about 30 nm, less than about 10 nm, less than about 3 nm, less than about 1 nm, or any value or interval thereof.

Nanoparticles for use in the present invention can be made employing a variety of biodegradable polymers used for controlled release formulations, as are well known in the art. Derivatized biodegradable polymers are also suitable for use in the present invention, including hydrophilic polymers (e.g., polyethylene glycol) attached to PLGA and the like.

Nanoparticle compositions and methods for making suitable nanoparticles that can be employed in the compositions and methods of the invention include those described in, e.g., those described in WO 97/04747 entitled “Drug Delivery Systems For Macromolecular Drugs”, U.S. Pat. No. 6,007,845 to Domb et al., U.S. Pat. No. 5,578,325 to Domb et al., U.S. Pat. No. 5,543,158 to Ruxandra et al., U.S. Pat. No. 6,254,890 to Hirosue et al., International Application No. PCT/US07/07927, filed Mar. 30, 2007, entitled “System for Targeted Delivery of Therapeutic Agents”, U.S. application Ser. No. 11/803,843, filed May 15, 2007, entitled “Polymers for Functional Particles”, the complete disclosure of which are incorporated by reference herein. Composition and methods for making nanoparticles of the invention are also described in Farokhazad et al., “Targeted nanoparticle-aptamer bioconjugates for cancer chemotherapy in vivo,” PNAS 103(16):6315-6320 (Apr. 18, 2006), and Farokhazad et al., “Nanoparticle-Aptamer Bioconjugates: A New Approach for Targeting Prostate Cancer Cells,” Cancer Research 64, 7668-7672 (Nov. 1, 2004), the complete disclosure of which are incorporated by reference herein

In some embodiments, nanoparticles (NPs) are built as aggregates of amphiphilic molecules that establish a hydrophobic core and expose hydrophilic moieties to the media. Nanoparticles can be prepared using the water-in-oil-in-water solvent evaporation procedure (double emulsion method) as described previously, e.g., in Gref, R. et al., Science 263, 1600-1603 (1994). Nanoparticles can also be prepared as described in Farokhazad et al., “Targeted nanoparticle-aptamer bioconjugates for cancer chemotherapy in vivo,” PNAS 103(16):6315-6320 (Apr. 18, 2006).

In some embodiments, a nanoparticle can be formed generally as follows. First one or more oligonucleotides of the invention are linked (e.g., by covalent attachment), to a amphiphilic molecule, e.g., a PLGA-PEG (poly(lactide-co-glycolide) and polyethylene glycol) diblock copolymer to form PLGA-PEG-oligonucleotide macromolecules. A nanoparticle is then formed by mixing the macromolecules with a plurality of amphiphilic molecules that can be the same or different, (e.g., PLGA-PEG diblock copolymers). Alternatively, the nanoparticle can be formed first without the olignucleotide, and the olignucleotide can be linked or attached thereafter.

Various modifications can be made as described, e.g., in U.S. application Ser. No. 11/803,843, filed May 15, 2007, entitled “Polymers for Functional Particles”, incorporated by reference herein. For example, the polymer or polymers can be mixed at varying ratios to form a series of particles having different properties, for example, different surface densities of oligonucleotide. For example, by controlling parameters such as PLGA molecular weight, the molecular weight of PEG, the oligonucleotide surface density, and the nanoparticle surface charge, very precisely controlled particles may be obtained.

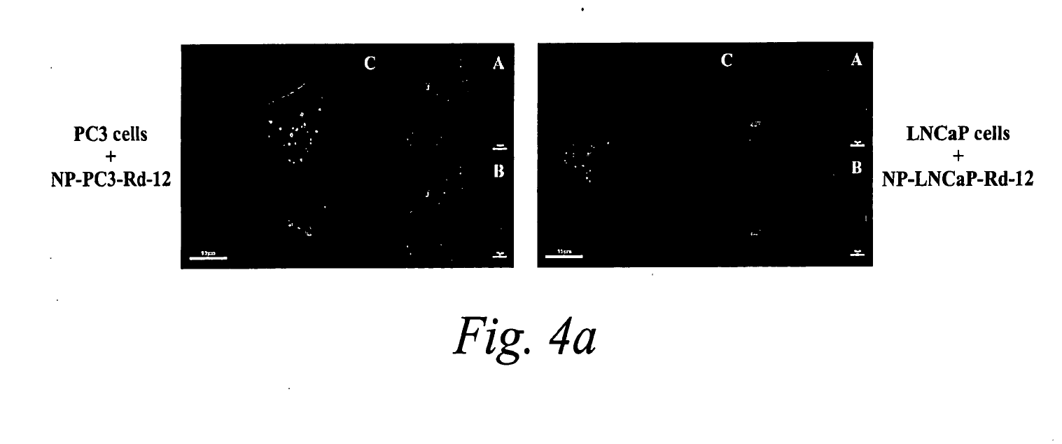

FIG. 3a is a depiction of a nanoparticle that can be used in accordance with the present invention. A digital image of an exemplary NP composition is shown in FIG. 3b. In some embodiments, the NPs employed include a number of diblock copolymers including a generally hydrophobic domain, e.g., PLGA, connected to a generally hydrophilic domain, e.g., a PEG, functionalized at the end with hydrophilic carboxylic acid moieties (PLGA-PEG-COOH).

Oligonucleotides can be associated with the nanoparticles by, e.g., conjugating the oligonucleotides to the hydrophilic block of a diblock copolymer included in the NP as described in the examples.

### Diagnostic or Imaging Agents

The identified oligonucleotides of the invention are suitable for being admixed, formulated, conjugated, or linked using known chemistries to facilitate the internalization of a diagnostic or imaging agent.

In another set of embodiments, the therapeutic agent is a diagnostic agent. For example, the therapeutic agent may be a fluorescent molecule; a gas; a metal; a commercially available imaging agents used in positron emissions tomography (PET), computer assisted tomography (CAT), single photon emission computerized tomography, X-ray, fluoroscopy, and magnetic resonance imaging (MRI); or a contrast agents. Non-limiting examples of suitable materials for use as contrast agents in MRI include gadolinium chelates, as well as iron, magnesium, manganese, copper, and chromium.

Examples of materials useful for CAT and x-ray imaging include, but are not limited to, iodine-based materials. As another example, the therapeutic agent may include a radionuclide, e.g., for use as a therapeutic, diagnostic, or prognostic agents. Among the radionuclides used, gamma-emitters, positron-emitters, and X-ray emitters are suitable for diagnostic and/or therapy, while beta emitters and alpha-emitters may also be used for therapy. Suitable radionuclides for forming use with various embodiments of the present invention include, but are not limited to, ¹²³1, ^{12S}I, ¹³⁰1, ¹³¹1, ¹³⁵1, ⁴⁷Sc, ⁷²As, ⁷²Sc, ⁹⁰Y, ⁸⁸Y, ⁹⁷Ru, ¹⁰⁰Pd, ^{101m}Rh, ^{U9}Sb, ¹²⁸Ba, ¹⁹⁷Hg, ²¹¹At, ²¹²Bi, ²¹²Pb, ¹⁰⁹Pd, ⁶⁷Ga, ⁶⁸Ga, ⁶⁷Cu, ⁷⁵Br, ⁷⁷Br, ^{99m}Tc, ¹⁴C, ¹³N, ¹⁵0, ³²P, ³³P, or ¹⁸F. The radionuclides may be contained within a particle (e.g., as a separate species), and/or form part of a macromolecule or polymer that forms associated with an oligonucleotide of the invention.

Therapeutic agents can be associated with the oligonucleotides of the invention employing any of the composition, methods or technologies described herein, e.g., nanoparticles or conjugates.

### Methods of Treatment

The present invention provides for both prophylactic and therapeutic methods of treating a subject requiring cell type specific delivery of a therapeutic agent. It is understood that “treatment” or “treating” as used herein, is defined as the application or administration of a therapeutic agent (e.g., a composition of the invention comprising a oligonucleotide docked to a chemotherapeutic agent) to a patient, or application or administration of a therapeutic agent to an isolated tissue or cell line from a patient, who has a disease or disorder, a symptom of disease or disorder or a predisposition toward a disease or disorder, with the purpose to cure, heal, alleviate, relieve, alter, remedy, ameliorate, improve or affect the disease or disorder, the symptoms of the disease or disorder, or the predisposition toward disease.

Administration of a prophylactic agent can occur prior to the manifestation of symptoms, such that a disease or disorder is prevented or, alternatively, delayed in its progression. These methods can be performed in vitro (e.g., by culturing the cell with the agent), in vivo (e.g., by administering the agent to a subject), or ex vivo.

In some embodiments, the methods are employed to treat a cancer (e.g., prostate cancer), a parasite (e.g., malaria), a viral infection (e.g., HIV), a hepatitis (e.g., hepatitis B).

Exemplary cancers include, but are not limited to, adrenocortical carcinoma; aids-related lymphoma; AIDS-related malignancies; anal cancer; bile duct cancer, extrahepatic; bladder cancer; bone cancer, osteosarcoma/malignant fibrous histiocytoma; cancers of the brain including among others brain stem glioma; cerebellar astrocytoma, cerebral astrocytoma/malignant glioma, ependymoma, medulloblastoma, supratentorial primitive neuroectodermal tumors, and visual pathway and hypothalamic glioma; breast cancer; bronchial adenomas/carcinoids; gastrointestinal carcinoid tumor; the various carcinomas including adrenocortical, islet cell and adenocarcinoma as well as carcinoma of unknown primary; central nervous system lymphoma; cervical cancer; other childhood cancers; clear cell sarcoma of tendon sheaths; colon cancer; colorectal cancer; cutaneous t-cell lymphoma; endometrial cancer; ependymoma; ovarian epithelial cancer; esophageal cancer; Ewing's family of tumors; extracranial germ cell tumor; extragonadal germ cell tumor; extrahepatic bile duct cancer; eye cancer, intraocular melanoma; retinoblastoma; gallbladder cancer; gastric (stomach) cancer; gastrointestinal carcinoid tumor; germ cell tumors including e.g., extracranial, extragonadal, and ovarian; gestational trophoblastic tumor; hairy cell leukemia; head and neck cancer; hepatocellular (liver) cancer; hypopharyngeal cancer; islet cell carcinoma (endocrine pancreas); Kaposi's Sarcoma; kidney cancer; laryngeal cancer; leukemias including e.g., acute lymphoblastic, acute myeloid, chronic lymphocytic, chronic myelogenous and hairy cell leukemias; lip and oral cavity cancer; liver cancer; non-small cell and small cell lung cancer; the various lymphomas, including e.g., AIDS-related, central nervous system and cutaneous T cell-lymphomas as well as Hodgkin's Disease, non-Hodgkin's lymphoma, and central nervous system lymphoma; waldenstrom's macroglobulinemia; malignant mesothelioma; malignant thymoma; medulloblastoma; melanoma; intraocular melanoma; merkel cell carcinoma; mesothelioma, malignant; metastatic squamous neck cancer with occult primary; multiple endocrine neoplasia syndrome; multiple myeloma/plasma cell neoplasm; mycosis fungoides; myelodysplastic syndrome; multiple myeloma; myeloproliferative disorders; nasal cavity and paranasal sinus cancer; nasopharyngeal cancer; neuroblastoma; oral cancer; oral cavity and lip cancer; oropharyngeal cancer; osteosarcoma/malignant fibrous histiocytoma of bone; ovarian cancer; ovarian epithelial cancer; ovarian germ cell tumor; ovarian low malignant potential tumor; pancreatic cancer; islet cell cancer; paranasal sinus and nasal cavity cancer; parathyroid cancer; penile cancer; pheochromocytoma; pineal and supratentorial primitive neuroectodermal tumors; pituitary tumor; plasma cell neoplasm/multiple myeloma; pleuropulmonary blastoma; prostate cancer; rectal cancer; renal cell (kidney) cancer (including among others progressive metastatic renal cell carcinoma (including among others clear cell and the collecting duct hamartoma variant); renal, pelvis and ureter transitional cell cancers; retinoblastoma; rhabdomyosarcoma; salivary gland cancer; malignant fibrous histiocytoma of bone; soft tissue sarcoma (including among others malignant mixed mullerian, liposarcoma and gist); sezary syndrome; skin cancer, including melanomas and Merkel cell cancer; small intestine cancer; soft tissue sarcoma; squamous neck cancer; stomach (gastric) cancer (including among others progressive metastatic gist); supratentorial primitive neuroectodermal tumors; testicular cancer; thymoma; thyroid cancer; transitional cell cancer of the renal pelvis and ureter; gestational trophoblastic tumor; cancers of an unknown primary site; unusual cancers of childhood; ureter and renal pelvis, transitional cell cancer; urethral cancer; uterine sarcoma (including among others malignant mixed mullerian); vaginal cancer; vulvar cancer and Wilms' Tumor.

### Pharmaceutical Compositions

A pharmaceutical composition of the invention is formulated to be compatible with its intended route of administration. For example, solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial compounds such as benzyl alcohol or methyl parabens; antioxidants such as ascorbic acid or sodium bisulfite; chelating compounds such as ethylenediaminetetraacetic acid; buffers such as acetates, citrates or phosphates and compounds for the adjustment of tonicity such as sodium chloride or dextrose. pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition will preferably be sterile and should be fluid to the extent that easy syringability exists. It will preferably be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal compounds, for example, parabens, chlorobutanol, phenol, ascorbic acid, thimerosal, and the like. In many cases, it will be preferable to include isotonic compounds, for example, sugars, polyalcohols such as manitol, sorbitol, sodium chloride in the composition. Prolonged absorption of the injectable compositions can be brought about by including in the composition an compound which delays absorption, for example, aluminum monostearate and gelatin.

### Kits

In another aspect, the invention provides kits for deriving one or more oligonucleotides for specific internal delivery to a target cell type. In one embodiment, kits of the invention comprise a plurality of oligonucleotides, and instructions for use. In one embodiment, the invention provides kits for deriving an oligonucleotide for specific internal delivery to cancer cells.

For example, in one embodiment, the kit can include a number of oligonucleotides that have been derived for specific internal delivery to one or more related cancer cell types. The kit can include instructions for employing the methods of the present invention to derive oligonucleotides specific for internal delivery to a related cancer cell type.

### EXEMPLIFICATION

The methods and compositions of this invention can be understood further by the examples that illustrate some of the processes by which these compositions are prepared and/or methods by which they are used. It will be appreciated, however, that these examples do not limit the invention. Variations of the invention, now known or further developed, are considered to fall within the scope of the present invention as described herein and as hereinafter claimed.

Throughout the examples, the following materials and methods were used unless otherwise stated.

### Materials and Methods

In general, the practice of the present invention employs, unless otherwise indicated, conventional techniques of chemistry, nucleic acid chemistry, recombinant DNA technology, molecular biology, biochemistry, cell culture and animal husbandry. See, e.g., DNA Cloning, Vols. 1 and 2, (D. N. Glover, Ed. 1985); Oligonucleotide Synthesis (M. J. Gait, Ed. 1984); Oxford Handbook of Nucleic Acid Structure, Neidle, Ed., Oxford Univ Press (1999); Sambrook, Fritsch and Maniatis, Molecular Cloning: Cold Spring Harbor Laboratory Press (1989); and Current Protocols in Molecular Biology, eds. Ausubel et al., John Wiley & Sons (1992).

### Cell Culture

The cell lines LNCaP, PC3, and RWPE-1 were obtained from the American Type Culture Collection (Manassas, Va.). The cell line PrEC was obtained from Cambrex (Hopkinton, Mass.). BPH-1 was obtained from Vanderbilt University Medical Center (Nashville, Tenn.). All of the cells were grown according to the manufacturer's specifications. LNCaP and BPH-1 cell lines were grown in RPMI 1640 medium, PC3 in Ham's F12K medium, RWPE-1 in KSF medium with EGF and BPX, and PrEC cell line in PrEGM and PrEBM medium. LNCaP, BPH, PC3, and RWPE-1 medium was supplemented with 100 units/mL aqueous penicillin G, 100 μg/mL streptomycin, and 10% fetal bovine serum was also added to LNCaP, BPH, PC3 medium.

### In Vitro Selection

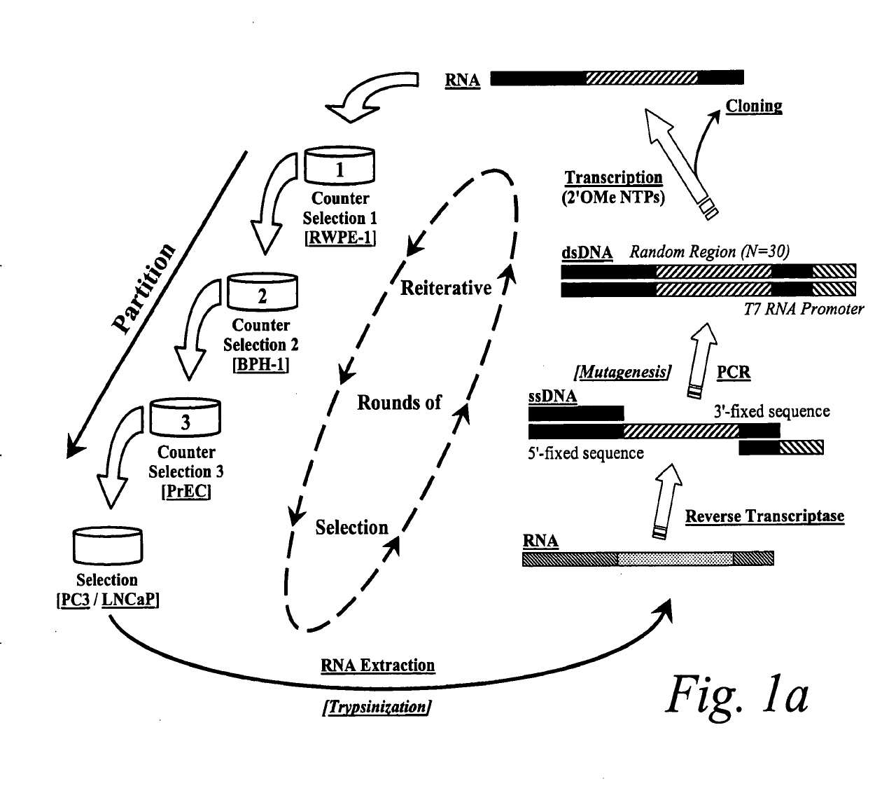

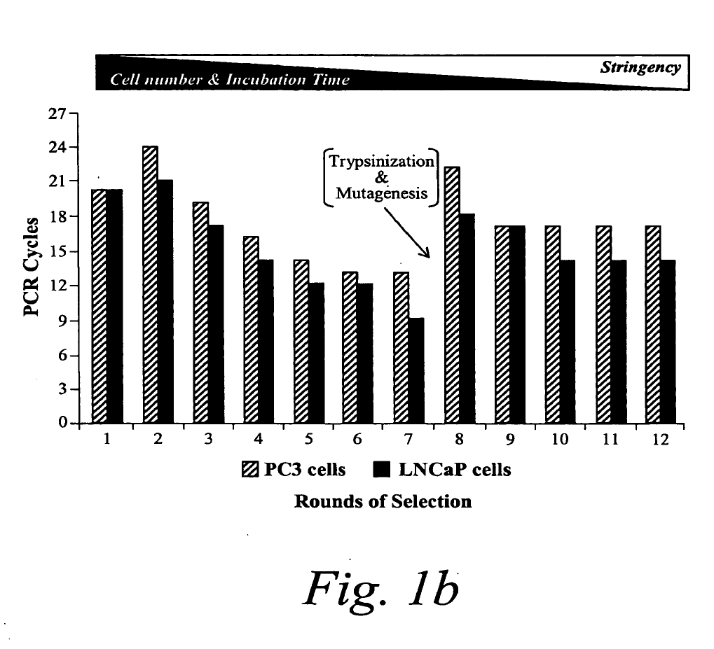

The selection protocol (FIG. 1a) was designed to enrich the amount of oligonucleotides which act as targeting agents in therapeutic devices. For example, degradation-resistant oligonucleotides that efficiently invade prostate cancer cells but leave healthy tissues unchallenged.

FIG. 1a is a schematic depiction of the in vitro selection of internalizing, disease-specific oligonucleotides. The general cycle protocol is as follows: the double-stranded DNA library was transcribed into 2′-O-methylated RNA, consecutively incubated with three counter-selective normal prostate cell strains (RWPE-1, BPH-1, and PrEC). Material not lost to the counter-selection was then presented and left to interact with either PC3 or LNCaP prostate cancer cells. After extensive washing, total RNA extraction, Reverse Transcription and PCR, a new cycle could be started. FIG. 1b is a graphical depiction of the progress of the selections: followed through the number of PCR-cycles necessary to amplify the selected material to reach a given amount. Stringency was increased by diminishing both the number of PC3 and LNCaP cells (10⁷ in Round-1, decreasing by 1-2×10⁶ cells per cycle, reaching 10⁶ for Round-12) and the incubation time (60 min for Rounds 1 and 2, three rounds of 45 min and 30 min until the end) of the selective step. After round 7, mutagenic PCR was used to explore the sequence-neighborhood of the selected libraries, and extensive trypsinization of the PC3 and LNCaP cells was applied to discard RNAs binding to the target cells without getting successfully internalized.

The DNA library (estimated 9×10¹⁴ unique sequences) 5′-CAT CGA TGC TAG TCG TAA CGA TCC NNN NNN NNN NNN NNN NNN NNN NNN NNN NNN C GAG AAC GTT TCT CTC CTC TCC CTA TAG TGA GTC GTA TTA-3′ (SEQ ID NO. 1) (N being any of the four nucleotides, with equal probabilities) (Operon Biotechnologies, Inc., Huntsville, Ala.) was amplified by PCR under standard conditions (template DNA=100 μg/μL; MgCl₂=50 mM; Tris=200 mM; KCl=500 mM; primers=10 μM; dNTPs=10 mM; enzyme=1 U/μL; and initial denaturation of 5 min at 95° C., followed by cycles of 95° C. for 30 sec, 65° C. for 30 sec, and 72° C. for 1 min, with final extension of 2 minutes at 72° C.), with the primers: Reverse-Primer 5′-CAT CGA TGC TAG TCG TAA CGA TCC-3′ (SEQ ID NO. 2) and Forward-Primer 5′-TAA TAC GAC TCA CTA TAG GGA GAG GAG AGA AAC GTT CTC G-3′ (SEQ ID NO. 3). The resultant pool of double-stranded DNA was precipitated and separated from unincorporated nucleotides by gel filtration.

The introduction of 2′-O-methyl groups can be beneficial because it can result in nuclease-resistant oligonucleotides that are safer, less expensive, and more amenable to industrial-scale production than other available options. Accordingly, 2′-O-Methyl-modified RNAs were obtained by overnight incubation at 37° C. of the reaction mixture: 200 nM template, 200 mM HEPES, 40 mM DTT, 10% PEG 8000, 0.01% Triton X-100, 2 mM spermidine, 1.0 mM each of 2′-O-methyl ATP, CTP, and UTP (Trilink, San Diego, Calif.); 1.0 mM GTP (Invitrogen Corporation, Carlsbad, Calif.), 5.5 mM MgCl₂, 1.5 mM MnCl₂, 10 U/ml inorganic pyrophosphatase (Sigma-Aldrich, St. Louis, Mo.), 200 nM T7 RNA polymerase (Epicentre Biotechnologies, Madison, Wis.) as previously described, e.g., in Burmeister, P. E. et al. Chem Biol 12, 25-33 (2005). The resultant oligonucleotides were precipitated by LiCl, incubated with RQ1 DNase (Promega, Madison, Wis.), and stored in water. Products were visualized on denaturing 10% PAGE. All reagents were purchased from Boston BioProducts (Worcester, Mass.), unless otherwise mentioned.

To aid the isolation of RNA that focuses on cancerous and not healthy cells, the RNA of every cycle of selection was made to interact with three different strains of normal prostate cells (RWPE-1, BPH-1, and PrEC) before being exposed to cultures of either LNCaP (androgen-dependent adenocarcinoma, derived from lymphnode metastasis and presenting the exclusively expressed Prostate Specific Antigen) or PC3 (androgen-independent adenocarcinoma, derived from bone metastasis) cells. The RNA library (1.5 nmol) was briefly denatured at 90° C. in 20 mL of EBSS (Invitrogen Corporation, Carlsbad, Calif.) with 1 mM magnesium chloride, cooled slowly and then warmed up to 37° C. before consecutive incubations with 10×10⁶ cells from each of the counter-selection cell-strains (RWPE-1, BPH-1, and PrEC) as described in FIG. 1a. After each incubation (60 minutes for the first 5 rounds of selection, 45 min afterwards), the unbound material was collected, filtered, and transferred to the next one. Oligonucleotides with affinity to features present in normal cells were iteratively weaned out of the population, enriching the fraction of sequences that relate specifically to recognition sites of the cancerous state.

The remaining pool was exposed to the selection cells, LNCaP or PC3, for an amount of time that varied throughout the selection: 60 min the first two rounds, 45 min for the next 3 rounds and 30 min for the rest of the selection. That is, after obtaining the “survivor sequences” of the three counter selections, the survivor sequences were incubated with PCa cells (PC3 or LNCaP) as described above. The cells were washed and the unbound sequences were aspirated several times. The cells were subsequently trypsinized, washed several times, and the RNA extracted.

Selected RNA was treated with RQ1 DNase (Promega, Madison, Wis.), before reverse-transcription and PCR amplification. The progress of the selection, measured by the number of PCR-cycles needed to amplify the chosen material for the next round (Rd, that is, the number of cycles needed to get the same amount of material), can be seen in FIG. 1b, with annotations for changes in stringency. The PCR products were purified, transcribed into modified RNA, treated with DNase and precipitated with LiCl, followed by ethanol, before being fed into the next selection cycle. During the selection, the number of PC3 and LNCaP cells exposed to the RNA library progressively decreased, starting with 10×10⁶ and diminishing by 1-2×10⁶ cells every other round until reaching 1×10⁶ for round 12.

### Mutagenesis

After 7 rounds of selection the material was amplified with 14 cycles of mutagenic PCR, (template DNA=25 μg/μL; MgCl₂=7 mM; Tris=10 mM; KCl=50 mM; primers=2 μM; dCTP & dTTP=1 mM; dGTP & dATP=0.2 mM; enzyme=0.05 U/μL; and MnCl₂=0.5 mM; annealing extended to 3 minutes) to introduce potentially beneficial mutations (roughly 0.79% mutations per position; 0.24 mutations per sequence). The resultant DNA pool was further treated as described for the other rounds.

### Cloning, Sequencing and Analysis of Selected Oligonucleotides

After 7 and 12 rounds of selection, sequences were cloned into the pCR-4 TOPO plasmid, using the TOPO-TA Cloning Kit (Invitrogen Corporation, Carlsbad, Calif.). Approximately 100 plasmids were sequenced for each round 7 population and around 600 for the round 12 pools.

Exemplary sequences are identified as set forth herein as SEQ ID NOs. 4-308.

Regions of possible sequence conservation were identified with the help of pile-ups and multiple-sequence alignments constructed employing the ClustalW program, which can be found at the web address http://www.ebi.ac.uk/clustalw/.

Sequencing of clones from rounds 7 (prior trypsinization and mutagenesis) and 12 revealed complex populations with no overtly dominant sequence. However, conservation of several short segments was evident. For example, the octamer “UGCGCGCG” was found in 4.7% (PC3) and 1.3% (LNCaP) of the clones from Rd 7 and, by Rd 12, it was presented in 14.5% (PC3) and 9.3% (LNCaP) sequences. Two hexamers of this octamer were even more notably abundant: “CGCGCG” appeared in 15.3% of PC3 and 11.7% of LNCaP clones from Rd 7, and in 48% of PC3 and 36.4% of LNCaP sequences of Rd 12; “GCGCGC” was found in 10.6% of PC3 and 5.2% of LNCaP samples from Rd 7 and 42.3% of PC3 and 35.2% of LNCaP Rd 12 clones. These frequencies are significant, because any given eight base-long tract should be expected in approximately 1 of every 2,850 (0.035%) of the random sequences in the unselected library and any specific hexamer would only emerge in roughly 1 of 164 (0.61%) of the random sequences.

In further studies, the following hexamers and septamers were also identified: CGCCUU (9.1% in PC3, and 13.8% in LNCaP); CGCGCC (13.6% in PC3, and 9.7% in LNCaP); GUUCGCG (4.8% in PC3, and 5.1% in LNCaP); UGUGUG (5.9% in PC3, and 4.7% in LNCaP); UGUGCGC (5.9% in PC3, and 7.3% in LNCaP).

### Fluorescent Labeling of Oligonucleotides

Oligonucleotides were labeled by covalently linking a fluorescent dye to their 3′-end and tracked by pseudoconfocal microscopy. Briefly, RNA was dissolved in DNase/RNase-free water (1 μg/μl) with sodium periodate (pH 4; 1 μl) to oxidize the 3′-terminus into an aldehyde (1 hour at 25° C.). Excess oxidant was removed by the addition of 2× sodium sulfite. The labeling was complete after adding excess of Alexa Fluor® 488 hydroxylamine (Invitrogen Corporation, Carlsbad, Calif.) and letting the condensation reaction run for 2 hours at 37° C. Finally, the labeled RNAs were extracted using standard ethanol-precipitation procedures.

Cellular Uptake of Selected Oligonucleotides

All cell lines (as described hereinabove) were grown at concentrations to allow 70% confluence in 24 h (i.e., LNCaP: 40,000 cells/cm²) and washed twice with prewarmed EBSS buffer before the addition of the nucleic acids. Before being combined with the cells, fluorescently-labeled RNA (5 μg) from the Rd-12 of each selection or the initial library was denatured at 90° C. for 3 min, cooled to room-temperature for 10 min, supplemented with magnesium chloride to reach 1 mM and then incubated at 37° C. for 10 min.

Cells were incubated for one hour with the labeled material then washed (with EBSS buffer), fixed (with 4% formaldehyde, followed by 0.1% triton-x), stained (with rhodamine-phalloidin, from Invitrogen Corporation, Carlsbad, Calif.), and mounted with DAPI (Vector Laboratories, Burlingame, Calif.). For pseudoconfocal imaging, cells were visualized with 1.4 numerical-aperature oil-immersion 25× or 60× objectives, and individual images were taken along their z-axis at 0.1-μm intervals with a computerized Zeiss Axiovert 200M microscope (Carl Zeiss Microimaging, Thornwood, N.Y.).

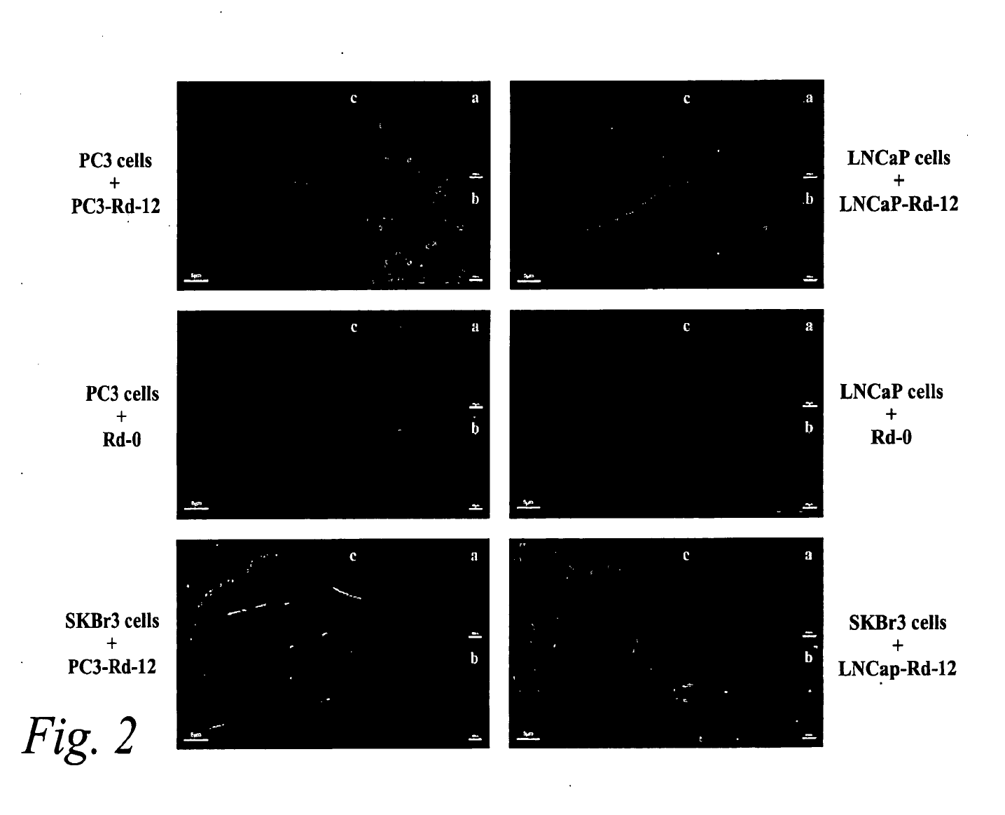

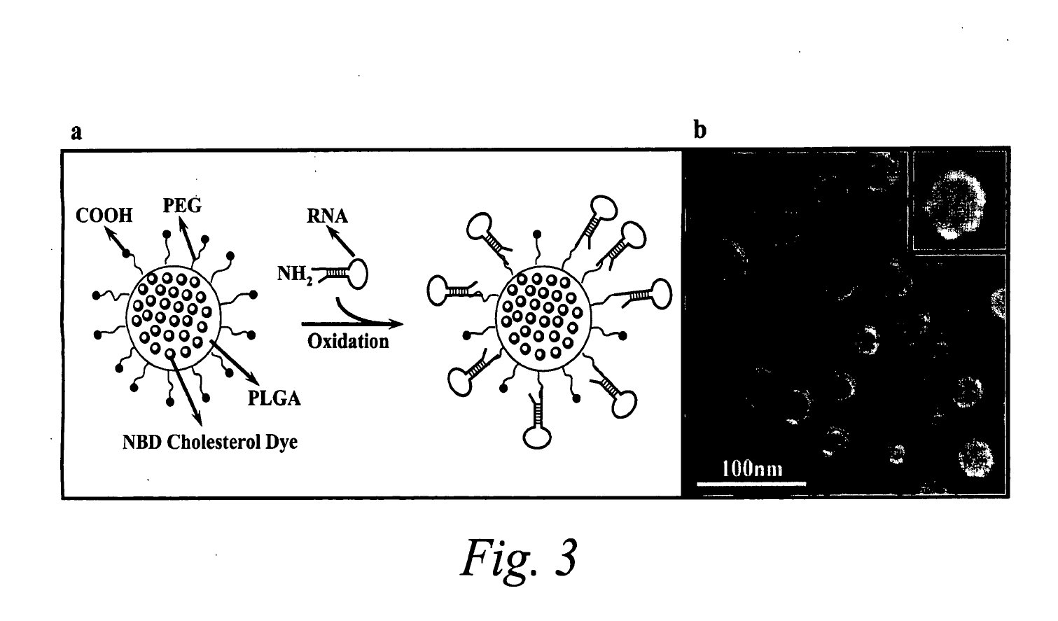

As can be seen in FIG. 2, the populations from the Rd-12 of both PC3 and LNCaP selections penetrated their respective target cells much more effectively than the initial random library did. Images taken in the z-section of these cells demonstrate that the fluorescent signal is indeed coming from within the cells. The images depict: (a) labeled oligonucleotides (FITC); (b) merged signals from the target cells of the nucleus (DAPI), cytoskeleton (Rhodamine Phalloidin), and the oligonucleotides (FITC); and (c) a single-cell close-up of the merged signal image.

The specificity of the selected oligonucleotides towards the intended cells was also evaluated by exposing the Rd-12 pools to cells from a variety of different strains: RWPE-1 (normal prostate epithelial), PrEC (normal prostate epithelial), BPH-1 (benign prostate hyperplasia), HUVEC (umbilical vein endothelial), HAEC (aortic endothelial), SKBR3 (breast cancer) and SKOV3 (ovarian cancer). No detectable signal was found inside these control cells, highlighting the potential feasibility of using the selected sequences for therapeutic purposes.

### MTT Cell Viability Assay

The feasibility of using the selected oligonucleotides as vehicles for internal and more effective delivery of doxorubicin into target cells was tested. Doxorubicin (Dox), a cytotoxic drug commonly used in chemotherapy that can dock into double-stranded portions of nucleic acids because of the stacking capacity of its many-ringed structure, was “loaded” onto oligonucleotides at a molar ratio of 1:1, Dox to RNA, e.g., as described in Bagalkot, V. et al., Angewandte Chemie (International ed 45, 8149-8152 (2006) (the drug concentration was 5 μM). MTT assays were performed essentially as previously described, e.g., in Akaishi, S. et al., The Tohoku Journal of Experimental Medicine 175, 29-42 (1995). Briefly, 100 μl aliquots of LNCaP or PC3 cells (5×10³ cells/mL) were seeded in 24-well plates (n=5), allowed to grow overnight and treated, for approximately one hour, with 100 μL of either a) nothing; b) Dox alone; c) Dox conjugated with the initial library; d) Dox conjugated to the Rd-12 population of the LNCaP selection; or e) Dox conjugated to the Rd-12 population of the PC3 selection; then washed and further incubated, in fresh media, for a total of 72 hours. The cells were then washed twice with EBSS, treated with 50 μL MTT solution for 4 hours, and lysed with MTT-detergent overnight. The absorbance was measured the following day using micro-plate reader at 570 nm.

The results indicate that free Dox is equipotent against PC3 and LNCaP cells but its effectiveness is enhanced when loaded onto the selected oligonucleotides, particularly when facing the specific target cells of each population.

### Nanoparticle Formation, Sizing and Shape

To further explore the versatility of the selected internalizing oligonucleotides we linked their 3′-end to the surface of nanoparticles bearing a fluorescent dye at their core, and incubated these complexes with PC3 and LNCaP prostate cancer cells. In short, nanoparticles (NPs) are built as aggregates of amphiphilic molecules that establish a hydrophobic core and expose hydrophilic moieties to the media. Nanoparticles were prepared using the nanoprecipitation method as described previously, e.g., in Farokhazad et al., PNAS 103, 6315-6320 (2006). In the presence of NBD cholesterol green dye, NPs were obtained that carried fluorescent labels at their core and could be covalently linked to the 3′-end of functionalized oligonucleotides (NH₂-RNA). FIG. 3a shows the composition of the nanoparticles (NPs) used, which are homogeneous in size (about 80 nm in diameter, in this case). The NPs used consist of PLGA domain connected to a PEG fragment functionalized at the end with hydrophilic carboxylic acid moieties (PLGA-PEG-COOH). To evaluate the size, surface charge and shape of the formed PLA-PEG-COOH nanoparticles, two complementary technologies were used. The size (nm) and surface charge (ζ potential in mV) were evaluated by Quasi-elastic laser light scattering with a ZetaPALS dynamic light scattering detector (Brookhaven Instruments Corporation, Holtsville, N.Y.; 15 mW laser, incident beam=676 nm). The nanoparticle conformation was determined by Transmission Electron Microscopy (TEM) where the nanoparticles were negatively stained with 2% Uranyl Acetate. Grids were viewed with a FEI Tecnai G2 Biotwin electron microscope operated at 80 KV and equipped with a high resolution digital camera, and can be seen in FIG. 3b. Transmission Electron Microscope (TEM) images of the 80 nm-diameter NP were obtained (±5 nm; polydispersity index ˜0.2). Size was measured both by TEM microscopy and Quasi-elastic laser light scattering, also used to evaluate their surface charge. Several fields were imaged at ×23K magnification and the size and shape of particles were measured using Improvision OpenLAb software.

### Formation of Fluorescent Nanoparticle-Oligonucleotide Conjugation

PLA-PEG-COOH nanoparticles encapsulating NBD cholesterol green dye (Invitrogen Corporation, Carlsbad, Calif.) were conjugated to the 3′ terminal of the oligonucleotides similarly to the labeling method described above. The RNA was oxidized to form aldehyde derivatives. Then, an excess of sodium sulphite (2×) was added to the solution to remove the excess oxidant. Then, five microliters of polymeric nanoparticle suspension (10 μg/μL in DNase RNase-free water) was incubated for 2 hours at room temperature with gentle stirring. The resulting bioconjugates were washed, resuspended, and preserved in suspension form in DNase RNase-free water.

### Cellular Uptake of Nanoparticle-Oligonucleotide Conjugates

All cell lines were grown at concentrations to allow 70% confluence, washed twice with pre-warmed binding buffer and incubated for one hour with the NP-oligonucleotide complexes in the presence of 1 mM magnesium chloride. The cells were then prepared for microscopy as indicated in the “Cellular uptake of selected oligonucleotides” subsection, above.

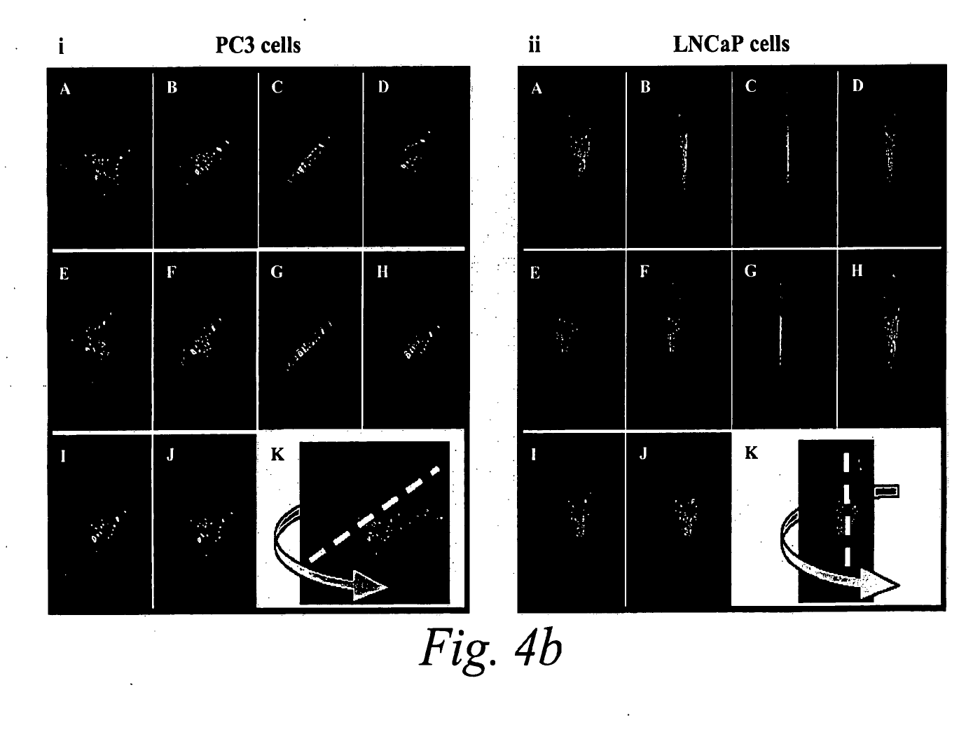

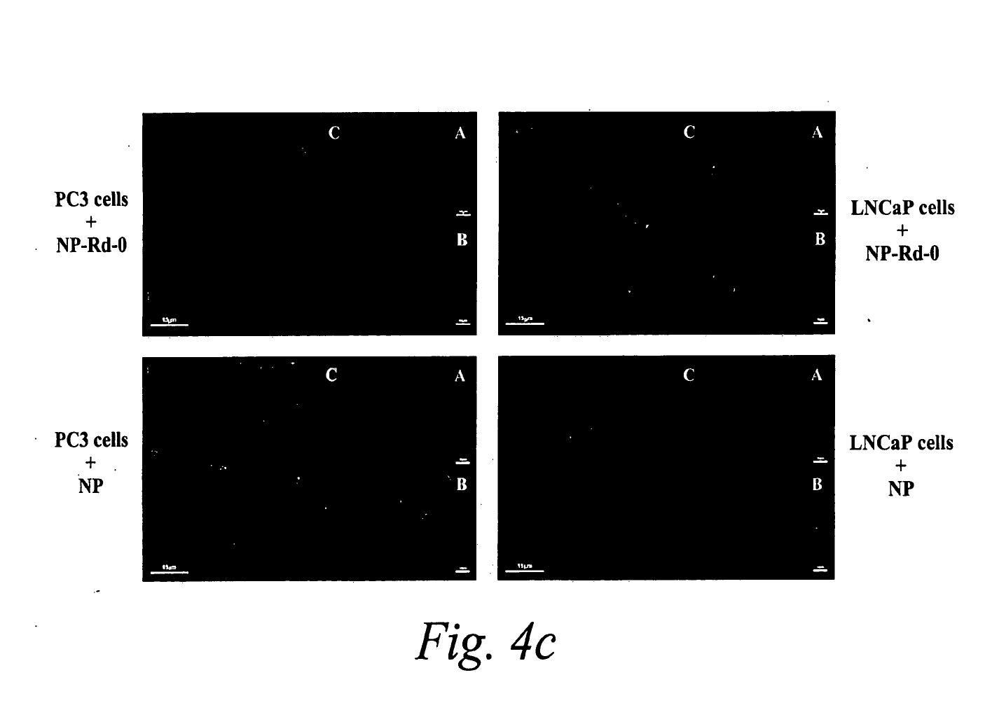

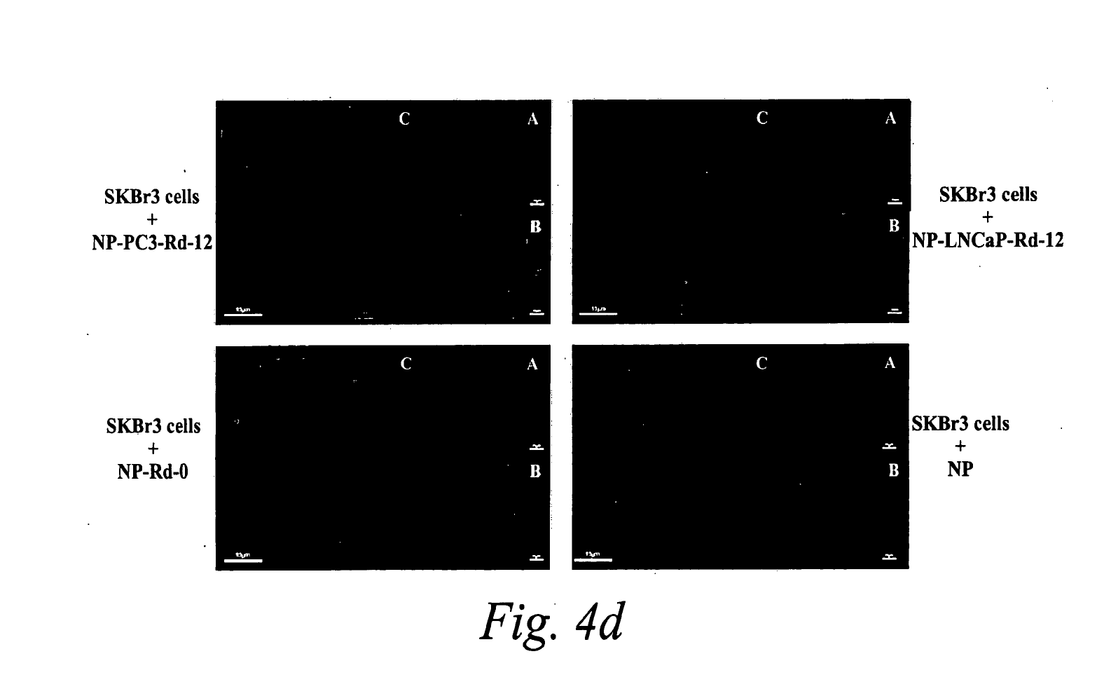

As can be seen in FIG. 4a, the presence of the selected oligonucleotides facilitated the invasion of cancer cells by the fluorescent NPs. Images were combined and deconvoluted to reconstruct a three-dimensional image of the cells for additional analysis. Fluorescent nanoparticles linked to Rd-12 populations entered PC3 and LNCaP cells are depicted as follows: subpanel A depicts fluorescent NP (NBD dye) linked to the indicated oligonucleotide population and incubated with the designated cells. Subpanel B depicts merged signals of the nucleus (DAPI), cytoskeleton (Rhodamine Phalloidin), and NP (NBD dye). Subpanel C depicts a single-cell close-up, labels as in B.

FIG. 4b shows the 3D-deconvolution of the images concerned, demonstrating that the signal of the NP-oligonucleotide complexes is coming from inside the cells. Tridimensional reconstruction of cell images confirm the nanoparticles are inside the cells. LNCaP and PC3 cells were grown on chamber slides and incubated with nanoparticles containing green NBD dye (22-(N-(7-nitrobenz-2-oxa-1,3-diazol-4-yl)amino)-23,24-bisnor-5-cholen-3β-ol) and linked to the Rd-12 populations of each selection. The cells were analyzed at 60× magnification along the z-axis at 0.2 μm intervals by fluorescent microscopy and approximately 150 individual images were combined to reconstruct each three-dimensional image of A through J show the same PC3 (i) or LNCaP (ii) cell, being rotated at 30-40 intervals; K demonstrates the rotation z-axis used in A through J images. The cell nuclei and the cytoskeleton are stained (4′,6-diamidino-2-phenylindole, DAPI) and (Rhodamine Phalloidin), respectively. The NBD at the core of the nanoparticle—RNA conjugates is also imaged.

Neither NPs alone nor those conjugated with the initial random library managed to reach the interior of the cells to any detectable levels (FIGS. 4c-d). The internalization of the nanoparticles requires the selected oligonucleotides and it only occurs with the target cells. No internalization was detectable when the nanoparticles were naked, linked to the initial RNA pool or accompanied by the Rd-12 populations but confronted to non-cognate cells. LNCaP, PC3 or SKBr3 cells, as noted, were presented to NP, NP derivatized with RNA from Rd-0 or NP presenting the selected oligonucleotides, as indicated. The sub-panels and dyes are as described in FIG. 5a.

### EQUIVALENTS

Those skilled in the art will recognize, or be able to ascertain using no more than routine experimentation, many equivalents to the specific embodiments of the invention described herein. Such equivalents are intended to be encompassed by the following claims.

### INCORPORATION BY REFERENCE

The contents of all references, patents, and patent applications cited throughout this application are hereby incorporated by reference.

1. A method for deriving an oligonucleotide for specific internal delivery to target cells, the method comprising:
providing a plurality of oligonucleotides; and
selecting at least once with target cells to provide a plurality of internalizing oligonucleotides,
wherein at least one oligonucleotide is derived that specifically internalizes into target cells.

2. The method according to claim 1, comprising counter-selecting at least once with a non-target cell type.

3. The method according to claim 1, wherein the method includes mutagenizing the plurality of internalizing oligonucleotides at least once.

4. The method according to claim 1, wherein the plurality of oligonucleotides are 2′-O-methyl-modified RNA oligonucleotides.

5. The method according to claim 1, wherein a plurality of oligonucleotides is derived that target a plurality of recognition sites.

6. The method according to claim 1, wherein the plurality of recognition sites are cell surface prostate cancer tumor antigens.

7. The method according to claim 2, wherein at least one of the non-target cell types is selected from the group consisting of non-cancer cells, normal prostate epithelial cells, RWPE-1 cells, PrEC cells, benign prostate hyperplasia cells, BPH-1 cells, endothelial cells, HUVEC cells, HAEC cells, and combinations thereof.

8. The method according to claim 1, wherein at least one of the target cell types is selected from the group consisting of cancer cells, prostate cancer cells, non-small cell lung cancer cells, breast cancer cells, ovarian cancer cells, PC3 cells, LNCaP cells, SKBR3 cells, SKOV3 cells, virus-infected cells, HIV-infected cells, malaria infected cells, hepatitis-infected cells, and combinations thereof.

9. The method according to claim 1, wherein the method comprises a plurality of consecutive incubations with at least one type of non-cancer cell and collecting unbound oligonucleotides.

10. The method according to claim 1, wherein the method comprises a plurality of consecutive incubations with at least one type of cancer cells and extracting a plurality of internalizing oligonucleotides from the cancer cells.

11. The method according to claim 2, further comprising: amplifying after counter-selecting or selecting at least once to provide a plurality of amplified oligonucleotides; and counter-selecting or selecting the plurality of amplified oligonucleotides at least once.

12. The method according to claim 2, wherein the method includes counter-selecting at least five times, and selecting at least three times.

13. An isolated oligonucleotide that specifically internalizes into at least one target cell type.

14. An isolated plurality of oligonucleotides that specifically internalizes into at least one target cell type.

15. The oligonucleotide or plurality of oligonucleotides according to claims 13 or 14, wherein at least one of the target cell types is selected from the group consisting of cancer cells, prostate cancer cells, non-small cell lung cancer cells, PC3 cells, LNCaP cells, virus-infected cells, HIV-infected cells, malaria infected cells, hepatitis-infected cells, and combinations thereof.

16. The oligonucleotide or plurality of oligonucleotides according to claims 13 or 14, wherein at least one oligonucleotide is capable of internalizing a therapeutic agent into a cancer cell.

17. The oligonucleotide or plurality of oligonucleotides according to claims 13 or 14, wherein at least one oligonucleotide is capable of internalizing a nanoparticle comprising a therapeutic agent into a cancer cell.

18. The oligonucleotide or plurality of oligonucleotides according to claims 13 or 14, wherein at least one oligonucleotide includes at least one sequence element selected from the group consisting of UGCGCGCG, CGCGCG, GCGCGC, CGCCUU, CGCGCC, GUUCGCG, UGUGUG, UGUGCGC, or the RNA or DNA complement of said sequence elements.

19. The plurality of oligonucleotides according to claims 13 or 14, wherein the plurality of oligonucleotides target a plurality of recognition sites.

20. The plurality of oligonucleotides of claim 19, wherein the plurality of recognition sites include at least one cell surface prostate cancer tumor antigen.

21. A composition for specific internal delivery of a therapeutic agent to target cells comprising:
a plurality of oligonucleotides according to claim 14; and
at least one therapeutic agents associated with at least one of the plurality of oligonucleotides,
wherein the composition is capable of specific internal delivery of the therapeutic agents to a target cell.

22. The composition of claim 21, wherein at least a portion of the therapeutic agents are docked to portion of the oligonucleotide.

23. The composition of claim 21, comprising a nanoparticle including a plurality of amphiphilic molecules that establish a hydrophobic core and hydrophilic moieties disposed about the core, and wherein at least a portion of the therapeutic agents are at least partially associated with the hydrophobic core and the oligonucleotide is associated with at least one hydrophilic moiety.

24. The composition according to claim 21, wherein the one or more therapeutic agents includes at least one agent selected from the group consisting of: a chemotherapeutic agent, a cytotoxic agent, and an antiviral agent.

25. A method of treating cancer comprising administering a composition according to claim 21, such that an effective amount of a therapeutic agent is delivered to a subject in need thereof and the cancer is treated.

26. The method of claim 25, wherein the cancer is prostate cancer.

27. A pharmaceutical formulation comprising the compositions claims 21-24 and a pharmaceutically acceptable carrier.

28. A method for determining nucleic sequence motifs associated with internalization of oligonucleotides into target cell type comprising:
providing a plurality of oligonucleotides;
counter-selecting at least once with a non-target cell type to provide a plurality of oligonucleotides that do not bind to features present in the non-target cell type;
selecting at least once with a target cell type to provide a plurality of internalizing oligonucleotides for the target cell type;
determining at least a portion of the nucleic acid sequence of the plurality of internalizing oligonucleotides for the target cell type; and
comparing the nucleic acid sequences, thereby determining common nucleic sequence motifs associated with internalization of oligonucleotides into a first cell type but not a second cell type.
